(19) United States
(12) Patent Application Publication
(10) Pub. No.: US 2022/0313650 A1
(43) Pub. Date: Oct. 6, 2022

(54) METHODS FOR TREATING NEURODEVELOPMENTAL DISORDERS

(71) Applicants: The Broad Institute, Inc., Cambridge, MA (US); Massachusetts Institute of Technology, Cambridge, MA (US)

(72) Inventors: Mario Arias Garcia, Cambridge, MA (US); Guoping Fong, Cambridge, MA (US); Zhanyan Fu, Cambridge, MA (US)

(73) Assignees: The Broad Institute, Inc., Cambridge, MA (US); Massachusetts Institute of Technology, Cambridge, MA (US)

(21) Appl. No.: 17/608,089

(22) PCT Filed: May 1, 2020
(86) PCT No.: PCT/US2020/031178

§ 371 Date: Nov. 1, 2021

Related U.S. Application Data
(60) Provisional application No. 62/842,357, filed on May 2, 2019.

(51) Int. Cl. A61K 31/34; A61K 31/496; A61P 25/16; C12N 15/09

(52) U.S. Cl. CPC A61K 31/34; A61K 31/496; A61P 25/16; C12N 15/09

(57) ABSTRACT

Provided herein are methods for treating a neurodevelopmental disorder using a group II metabotropic glutamate receptor modulator.

### CROSS REFERENCE TO RELATED APPLICATIONS

This application claims the benefit under 35 U.S.C. § 119(e) of U.S. Provisional Application No. 62/842,357, filed May 2, 2019, entitled “Methods for Treating Neurodevelopmental Disorders,” the entire disclosure of which is hereby incorporated by reference.

### FEDERALLY SPONSORED RESEARCH

This invention was made with government support under Grant No. NS098505 awarded by the National Institutes of Health. The government has certain rights in the invention.

### FIELD OF THE INVENTION

The present disclosure relates to treatment of neurodevelopmental disorders by administering an effective amount of a group II metabotropic glutamate receptor modulator.

### BACKGROUND

The thalamic reticular nucleus (TRN), a thin shell of GABAergic neurons that surrounds the dorsal thalamus, receives input from cortical and subcortical regions, and provides the major inhibition to thalamocortical (TC) neurons. The TRN plays a key role in sensory processing, arousal, and cognition. TRN dysfunction has been implicated in various behavioral deficits in neurodevelopmental disorders such as autism spectrum disorder (ASD), attention-deficit hyperactivity disorder (ADHD), and schizophrenia.

### SUMMARY

The present disclosure is based, at least in part, on the finding that modulation of group II metabotropic glutamate receptors can rescue behavioral deficits in a mouse model of thalamic reticular nucleus (TRN) dysfunction. The present disclosure provides a therapeutic strategy for novel treatments of neurodevelopmental disorders, psychiatric disorders, or sleep disorders. The present disclosure provides, in some embodiments, methods for treating a neurodevelopmental disorder. Some aspects of the present disclosure provide administering to a subject in need thereof an effective amount of a group II metabotropic glutamate receptor modulator. The present disclosure provides methods for treating attention-deficit hyperactivity disorder (ADHD). Some aspects of the present disclosure provide administering an effective amount of a group II metabotropic glutamate receptor modulator to a subject who has been diagnosed with ADHD.

The present disclosure provides, in some embodiments, a method for treating a psychiatric disorder. Some aspects of the present disclosure provide administering to a subject in need thereof an effective amount of a group II metabotropic glutamate receptor modulator. In some embodiments, the psychiatric disorder is not schizophrenia.

The present disclosure provides, in some embodiments, a method for treating a sleep disorder. Some aspects of the present disclosure provide administering to a subject in need thereof an effective amount of a group II metabotropic glutamate receptor modulator.

The present disclosure provides, in some embodiments, methods of improving sleep quality. The present disclosure provides, in some embodiments, methods of increasing sleep duration. The present disclosure provides, in some embodiments, methods of improving sleep quality and/or increasing sleep duration. Some aspects of the present disclosure provide administering to a subject in need thereof an effective amount of a group II metabotropic glutamate receptor modulator.

The present disclosure provides, in some embodiments, methods for treating a neurodevelopmental disorder involving administering an effective amount of a group II metabotropic glutamate receptor modulator to a subject who has been identified as having a loss-of-function mutation in the Patched Domain Containing 1 (PTCHD1) gene.

The present disclosure provides, in some embodiments, methods for treating a neurodevelopmental disorder involving determining whether a subject has a loss-of-function mutation in the Patched Domain Containing 1 (PTCHD1) gene. In some embodiments, the present disclosure provides administering an effective amount of a group II metabotropic glutamate receptor modulator to the subject if the subject has a loss-of-function mutation in the PTCHD1 gene.

In some embodiments, the group II metabotropic glutamate receptor modulator is a mGluR_{2/3} agonist. In some embodiments, the mGluR_{2/3} agonist is LY354740. In some embodiments, the mGluR_{2/3} agonist is MGS0028. In some embodiments, the mGluR_{2/3} agonist is LY379268. In some embodiments, the mGluR_{2/3} agonist is LY2934747. In some embodiments, the mGluR_{2/3} agonist is LY2969822. In some embodiments, the mGluR_{2/3} agonist is LY404040. In some embodiments, the mGluR_{2/3} agonist is LY404039. In some embodiments, the mGluR_{2/3} agonist is LY2140023.

In some embodiments, the group II metabotropic glutamate receptor modulator is a mGluR₃-specific modulator. In some embodiments, the mGluR₃-specific modulator is a mGluR₃ agonist. In some embodiments, the mGluR₃-specific modulator is a mGluR₃ positive allosteric modulator (PAM). In some embodiments, the mGluR₃ agonist is LY2794193. In some embodiments, the mGluR₃ PAM is DT011088. In some embodiments, the mGluR₃ PAM is Mavalon-63 PAM. In some embodiments, the mGluR₃ PAM is Mavalon-207 PAM.

In some embodiments, the subject is a human subject.

In some embodiments, the neurodevelopmental disorder is attention-deficit hyperactivity disorder (ADHD). In some embodiments, the neurodevelopmental disorder is a learning disorder. In some embodiments, the neurodevelopmental disorder is a motor disorder. In some embodiments, the neurodevelopmental disorder is a tic disorder. In some embodiments, the neurodevelopmental disorder is a speech disorder. In some embodiments, the neurodevelopmental disorder is a genetic disorder. In some embodiments, the neurodevelopmental disorder is a neurotoxicants-related disorder. In some embodiments, the neurodevelopmental disorder is intellectual disability (ID). In some embodiments, the neurodevelopmental disorder is an autism spectrum disorder (ASD). In some embodiments, the subject is a human subject having, suspected of having, or at risk of developing a neurodevelopmental disorder as disclosed herein. In some embodiments, the subject is a human subject having, suspected of having, or at risk of having, a loss-of-function mutation in the Patched Domain Containing 1 (PTCHD1) gene.

In some embodiments, a subject is identified as having a loss-of-function mutation in the PTCHD1 gene based on a polymerase chain reaction (PCR) assay. In some embodiments, determining whether a subject has a loss-of-function mutation in the PTCHD1 gene comprises conducting a polymerase chain reaction (PCR) assay. In some embodiments, a subject is identified as having a loss-of-function mutation in the PTCHD1 gene based on a nucleic acid microarray assay. In some embodiments, determining whether a subject has a loss-of-function mutation in the PTCHD1 gene comprises conducting a nucleic acid microarray assay. In some embodiments, a subject is identified as having a loss-of-function mutation in the PTCHD1 gene based on an immunohistochemical assay. In some embodiments, determining whether a subject has a loss-of-function mutation in the PTCHD1 gene comprises conducting an immunohistochemical assay.

In some embodiments, a subject is identified as having a loss-of-function mutation in the PTCHD1 gene based on an immunoblotting assay. In some embodiments, determining whether a subject has a loss-of-function mutation in the PTCHD1 gene comprises conducting an immunoblotting assay. In some embodiments, a subject is identified as having a loss-of-function mutation in the PTCHD1 gene based on a flow cytometry assay. In some embodiments, determining whether a subject has a loss-of-function mutation in the PTCHD1 gene comprises conducting a flow cytometry assay. In some embodiments, the loss-of-function mutation in the PTCHD1 gene is an insertion. In some embodiments, the loss-of-function mutation in the PTCHD1 gene is a deletion. In some embodiments, the loss-of-function mutation in the PTCHD1 gene is a substitution.

In some embodiments, the present methods further comprise administering to the subject an additional therapeutic agent. In some embodiments, the additional therapeutic agent can comprise one or more of metformin, memantine, flumazenil, meclofenoxate, risperidone, carbamazepine, sodium valproate, lamotrigine, lithium carbonate, methylphenidate, procyclidine, ferrous fumarate+vitamins+lactulose+cod liver oil+various skin ointments, clobazam+lorazepam, rectal diazepam+buccal midazolam, omega-3 fatty acids+inositol, n-acetylcysteine, intranasal oxytocin, memantine hydrochloride, lovastatin, BPN14770, dronabinol, THC, 18F-AV-1451, ketamine, midazolam, d-cycloserine, rivaroxaban, acetylsalicylic acid, metformin amisulpride, bromocriptine, acetazolamide, antipsychotics including risperidone, aripiprazole, ziprasidone, SSRIs including fluoxetine, citalopram, escitalopram, stimulants including methylphenidate, alpha-2-adrenergic agonists including clonidine, guanfacine, propranolol, beta-blockers, primidone, clonazepam, diazepam, lorazepam, alprazolam, dopamine antagonists, aripiprazole, antipsychotics including clonidine, risperidone, olanzapine, ziprasidone, haloperidol, fluphenazine, pimozide, tetrabenazine, guanfacine, skeletal muscle relaxants including baclofen, benzodiazepines including clonazepam, neuromuscular blockers including onabotulinumtoxinA, amantadine, cyclosporine A, donepezil, glyburide, lithium, methylphenidate, minocycline, progesterone, rivastigmine, simvastatin, lithium, fenobam, γ-aminobutyric acid agonists, analgesics including acetametophin and codeine, morphine, ibuprofen, naproxen, antidysrthymics including digoxin, furosemide, hydrochlorothiazide, metolazone, memantine, antipsychotics including aripiprazole, asenapine, brexpiprazole, buspirone, cariprazine, chlorpromazine hydrochloride, clozapine, haloperidol, iloperidone, loxapine, lumateperone, lurasidone hydrochloride, molindone hydrochloride, olanzapine, paliperidone, perphenazine, prochlorperazine, quetiapine, risperidone, thiothixene, trifluoperazine, ziprasidone, androgens including testosterone,methyltestosterone, fluoxymesteron, gonadotrophins including chorionic gonadotropin and follitropin, teriparatide, stimulants including methamphetamine, methylphenidate, dexmethylphenidate, amphetamine, dextroamphetamine, Lisdexamfetamine, isdexamfetamine dimesylate, bupropion, venlafaxine, imipramine, risperidone, lithium and methylphenidate, carbamazepine, quetiapine, anticonvulsants such as citalopram, escitalopram, fluoxetine, paroxetine, sertraline, central alpha-2 adrenergic agonists including clonidine, guanfacine, and norepinephrine reuptake inhibitors including atomoxetine.

Each of the limitations of the invention can encompass various embodiments of the invention. It is, therefore, anticipated that each of the limitations of the invention involving any one element or combinations of elements can be included in each aspect of the invention. This invention is not limited in its application to the details of construction and the arrangement of components set forth in the following description or illustrated in the drawings. The invention is capable of other embodiments and of being practiced or of being carried out in various ways. Also, the phraseology and terminology used herein is for the purpose of description and should not be regarded as limiting. The use of “including,” “comprising,” or “having,” “containing,” “involving,” and variations of thereof herein, is meant to encompass the items listed thereafter and equivalents thereof as well as additional items.

### BRIEF DESCRIPTION OF THE DRAWINGS

The following drawings form part of the present specification and are included to further demonstrate certain aspects of the present disclosure, which can be better understood by reference to one or more of these drawings in combination with the detailed description of specific embodiments presented herein. The accompanying drawings are not intended to be drawn to scale. The drawings are illustrative only and are not required for enablement of the disclosure. For purposes of clarity, not every component may be labeled in every drawing. In the drawings:

FIG. 1 is a schematic diagram of the thalamocortical circuit.

FIG. 2 is an image of brain tissue showing that schizophrenia and ASD risk genes are expressed in the TRN.

FIG. 3A is a schematic diagram showing single nucleus RNA-sequencing (sNuc-Seq) experiments. TRN tissues were labeled with TdTomato, micro-dissected in a series of 200 μm sagittal sections, and extracted for nuclei from PV-td Tomato mice. The nuclei were then sorted into single wells for RNA sequencing.

FIG. 3B is a 2D t-SNE plot of embedding of single nuclei showing expression of Grm2 and Grm3.

FIG. 3C is an image of the TRN showing expression of Pvalb and Grm3.

FIG. 3D is a Western blot showing expression of mGluR₃ in TRN tissue.

FIG. 3E depicts representative traces showing an increase in the number of rebound burst firings in the presence (bottom) and absence (top) of Mavalon 63 after somatic hyperpolarization pulse in WT TRN neurons.

FIG. 3F is a graph summarizing the changes in the number of rebound bursting at different membrane holding potentials ranging from −80 mV to −50 mV. Significant changes were detected at a membrane holding potentials between −70 mV and −60 mV in the presence of Mavalon 63 (squares, n=12 cells, paired t-test, * p<0.05).

FIG. 4A depicts representative traces showing an increase in the number of rebound burst firings in the presence of the mGluRII agonist LY379268 (1 μM) after somatic hyperpolarization pulse.

FIG. 4B depicts representative traces showing that ML337 (5 μM) reduces the repetitive burst firings after somatic hyperpolarization pulse.

FIG. 4C is a graph summarizing the changes in the number of rebound bursting in different membrane holding potentials ranging from −80 mV to −50 mV. Significant changes were detected in a membrane holding potentials between −70 mV and −60 mV in the presence of LY379268 (n=13).

FIG. 4D depicts graphs showing that the number of rebound burst firing was reduced after the bath application of ML337 (n=11). Increased rebound burst firing by LY379268 was occluded in the presence of ML337 (n=7). *P<0.05, paired-t test.

FIG. 4E depicts representative T type and SK current traces in the presence of the SK channel blocker apamin (100 nM).

FIG. 4F is a graph summarizing the changes in the current mediated by T in the presence of LY379268 (n=8 cells, paired t-test, * p<0.05).

FIG. 4G is a graph summarizing the changes in the current mediated by SK in the presence of LY379268 (n=8 cells, paired t-test, * p<0.05).

FIG. 5A depicts the injection site of mcherry-dflox.hChR2.AAV1 in layer 5/6 of the primary somatosensory cortex in Nex-Cre mouse, and virus injection in (VB) in Vlgut2-Cre mouse.

FIG. 5B shows representative recordings of optogenetic evoked EPSCs in patched TRN neurons (20 Hz), before (top panel) and after (middle panel) the bath application of LY379268 (1 μM). The amplitude was significantly changed as well as the paired-pulse ratio.

FIG. 5C depicts recordings and graphs showing that LY379268 did not affect the thalamo-TRN evoked EPSCs in patched TRN neurons. * P<0.05, paired-t-test.

FIG. 5D depicts representative paired pulse ratio (PPR) recording traces of optogenetically evoked two consecutive EPSCs of TRN neurons, before (baseline, top) and after (bottom) the bath application of AZD8529.

FIG. 5E depicts representative PPR recording traces of TRN neurons before (baseline, top) and after (bottom) the bath application of Mavalon 63.

FIG. 5F is a graph showing the size of PPR was significantly increased after the bath perfusion of AZD8529.

FIG. 5G is a graph showing the size of PPR was not significantly increased after the bath perfusion of Mavalon 63.

FIG. 5H depicts representative optogenetically evoked PPR recording traces of TRN neurons, before (baseline, top) and after (bottom) the bath application of AZD8529.

FIG. 5I depicts representative optogenetically evoked PPR recording traces of TRN neurons, before (baseline, top) and after (bottom) the bath application of Mavalon63.

FIG. 5J is a graph showing no significant changes in the size of PPR after the bath perfusion of AZD8529.* P<0.05, paired-t-test.

FIG. 5K is a graph showing no significant changes in the size of PPR after the bath perfusion of Mavalon 63. * P<0.05, paired-t-test.

FIG. 6A shows representative traces of the rebound burst firing in a TRN neuron in Ptchd1 KO mice before (upper panel) and after the bath application of the group II metabotropic glutamate receptor modulators LY379268 (middle panel) or Mavalon 63 (lower panel).

FIG. 6B shows a graph summarizing the maximum effect of LY379268 on the number of rebound bursts among individual Ptchd1 KO TRN neurons, plotting number of rebound bursts observed before (control) and after the treatment with LY379268.

FIG. 6C shows a graph summarizing the maximum effect of Mavalon 63 on the number of rebound bursts among individual Ptchd1 KO TRN neurons, plotting number of rebound bursts observed before (control) and after the treatment with Mavalon 63.

FIG. 7A shows representative rebound bursting firing of Ptchd1 KO TRN neuron as a response of hyperpolarizing square pulse (upper panel), and after bath application of LY379268 (lower panel; 1 μM), which significantly increased the number of rebound burst firings.

FIG. 7B shows a graph summarizing the changes in the number of rebound bursting at various membrane holding potentials ranging from −85 mV to −50 mV. Significant changes were found between −72 mV and −67 mV holding potential in the presence of LY379268 (n=8) *P<0.05, paired-t-test.

FIG. 7C depicts representative traces showing an increase in the number of rebound burst firings of TRN neurons from WT mice in the presence (bottom) and absence (top) of LY379268 after somatic hyperpolarization pulse.

FIG. 7D is a graph summarizing the changes in the number of rebound bursting at different membrane holding potentials ranging from −80 mV to −50 mV. Significant changes were detected at membrane holding potentials as indicated in the presence of LY379268 (squares, paired t-test, * p<0.05).

FIG. 7E depicts the timeline of treatment of vehicle and LY379268 and schematic representation of the simultaneous EEG/EMG recordings and representative traces of EEG and EMG recordings for Non-REM sleep and awake status.

FIG. 7F is a graph showing LY379268 administration increased the length of NREM sleep bout in WT mice for 3 h recordings.

FIG. 7G is a graph showing a significant increase in the length of NREM sleep bout in Ptchd1 KO mice for 3 h recordings (paired t-test, n=6 animals, *P<0.05)

FIG. 7H depicts graphs showing the total distance traveled for WT mice treated with vehicle (saline) and three dosages of LY379268 as indicated (i.p.) for 90 minutes (n=7, *P<0.05, unpaired t-test.)

FIG. 7I depicts graphs summarizing the total distance traveled comparing vehicle and LY379268 treatment Ptchd1 KO mice for 90 minutes. (n=7 *P<0.05, unpaired-t-test).

FIG. 8A is an image of a brain slice showing the TRN.

FIG. 8B depicts representative traces from WT and Ptchd1 KO mice indicating impaired repetitive bursting in Ptchd1 KO TRN neurons.

FIG. 9A is a schematic representation of the simultaneous EEG/EMG recordings and representative traces of EEG and EMG recordings for Non-REM sleep and awake status.

FIG. 9B is a plot showing the increased Non-REM sleep percentage and increased mean Non-REM bout after treatment with LY379268.

FIG. 9C depicts data from a spectral analysis of normalized power of EEG showing the increased delta rhythm (1-4 Hz) before and after LY379268, 5 mg/Kg, I.P. injection.

FIG. 9D is a graph showing Mavalon 63 (10 mg/kg, ip) increased the length of NREM sleep bout in Ptchd1 KO mice for 12 h recordings, but no change was observed for wild type (WT) mice. Paired t-test, n=5-6 animals, *P<0.05.

FIG. 10A is a graph showing the distance travelled over time for WT mice intraperitoneally injected with vehicle (saline) or LY379268 (5 mg/kg).

FIG. 10B is a graph showing the distance travelled over time for Ptchd1 KO mice intraperitoneally injected with vehicle (saline) or LY379268 (5 mg/kg).

FIG. 10C is a graph summarizing the total distance traveled comparing vehicle and LY379268 injection in WT and Ptchd1 KO mice for 90 minutes. (n=6 WT, Vehicle. n=7 WT, LY379268. n=9 KO, Vehicle. n=8 KO, LY379268). *P<0.05, unpaired-t-test.

FIG. 11A is a schematic depicting sound discrimination task (GO/NOGO) experiments.

FIG. 11B is a graph summarizing behavioral performance of WT mice (circles) and Ptchd1 KO mice (squares). KO mice displayed reduced percentages of correct responses in the presence of auditory stimuli embedded with noise, which were rescued to control levels after treatment with 5 mg/kg LY379268. (n=4 WT and 5 KO mice).

FIG. 11C depicts a summary of percentage of correct responses for WT mice in both pure tone trials (left) and noise trials (auditory stimuli embedded with noise, right).

FIG. 11D depicts a summary of percentage of response accuracy in both pure tone and noise trials for Ptchd1 KO mice (n=4 for WT and 5 for KO mice).

FIG. 12A is a graph showing the total distance traveled for WT mice treated with vehicle (saline) and three dosages of Mavalon 63 (5 mg/Kg, 10 mg/Kg, 30 mg/Kg, i.p.) for 90 minutes.

FIG. 12B is a graph showing the total distance traveled for Ptchd1 KO mice treated with vehicle (saline) and three dosages of Mavalon 63 (5 mg/Kg, 10 mg/Kg, 30 mg/Kg, i.p.) for 90 minutes.

FIG. 12C is a graph comparing the vehicle and Mavalon 63 administration in WT and Ptchd1 KO mice. (n=6-9, Kolmogorov-Smirnov test).

FIG. 12D is a graph showing significant changes in the size of paired-pulse ratio (PPR) after the bath perfusion of LY379268 The total distance traveled for WT mice treated with vehicle (saline) and three dosages of AZD8529 (i.p.) for 90 minutes.

FIG. 12E is a graph summarizing the total distance traveled for Ptchd1 KO mice treated with vehicle (saline) and three dosages of AZD8529 (10 mg/Kg, 20 mg/Kg, 50 mg/Kg i.p.).

FIG. 12F is a graph comparing the vehicle and AZD8529 administration in WT and Ptchd1 KO mice. (n=6-9, *P<0.05, Kolmogorov-Smirnov test).

FIG. 13A is a graph showing representative spontaneous excitatory postsynaptic current (sEPSCs) in the presence of GABAergic synaptic transmission blocker PTX with or without the bath perfusion of LY379268 (1 uM) in wild type (WT) mice.

FIG. 13B is a graph showing representative spontaneous excitatory postsynaptic current (sEPSCs) in the presence of GABAergic synaptic transmission blocker PTX with or without the bath perfusion of LY379268 in Ptchd1 KO mice.

FIG. 13C is a graph summarizing the changes in the frequency of sEPSCs in the presence of LY379268 (squares, n>6).

FIG. 13D) is a graph summarizing the changes in the peak amplitude of sEPSCs in the presence of LY379268 (squares, n>6).

FIG. 13E is a graph showing representative miniature excitatory postsynaptic current (mEPSCs) in the presence of GABAergic synaptic transmission blocker PTX and action potential blocker TTX with or without the bath perfusion of LY379268 (1 uM) in wild type (WT) mice.

FIG. 13F is a graph showing representative miniature excitatory postsynaptic current (mEPSCs) in the presence of GABAergic synaptic transmission blocker PTX and action potential blocker TTX with or without the bath perfusion of LY379268 in Ptchd1 KO mice.

FIG. 13G is a graph summarizing the changes in the frequency of mEPSCs in the presence of LY379268 (squares, n>6)

FIG. 13H) is a graph summarizing the changes in the peak amplitude of mEPSCs in the presence of LY379268 (squares, n>6). p<0.05, paired-t-test.

### DETAILED DESCRIPTION OF THE INVENTION

Neurodevelopmental disorders are multifaceted conditions characterized by impairments in cognition, communication, behavior and/or motor skills. Such behavioral deficits may be caused by thalamic reticular nucleus (TRN) dysfunction. The present disclosure is based, at least in part, on the finding that mice lacking the Patched Domain Containing 1 (PTCHD1) gene exhibited TRN dysfunction and behavioral deficits associated with neurodevelopmental disorders that could be rescued by administering a group II metabotropic glutamate receptor modulator. Accordingly, the present disclosure provides methods for treating a subject having a neurodevelopmental disorder using an effective amount of a group II metabotropic glutamate receptor modulator.

### Methods of Treating a Neurodevelopmental Disorder, a Psychiatric Disorder, or a Sleep Disorder

Aspects of the present disclosure provide methods for treating a neurodevelopmental disorder using an effective amount of a group II metabotropic glutamate receptor modulator. As used herein, a “neurodevelopmental disorder” refers to any disorder in which the development of the central nervous system (CNS) is disturbed. In some embodiments, the neurodevelopmental disorder is associated with TRN dysfunction. In some embodiments, the neurodevelopmental disorder is not a schizophrenia disorder. In some embodiments, the neurodevelopmental disorder is not associated with disorders or symptoms associated with sleep spindle deficits.

Examples of neurodevelopmental disorders include, but are not limited to, intellectual disability (ID), intellectual and developmental disability (IDD) or mental retardation, motor disorders, autism spectrum disorder (ASD), attention-deficit hyperactivity disorder (ADHD), specific learning disorders, tic disorders, traumatic brain injury, communication, speech and language disorders, genetic disorders, disorders associated with neurotoxicants, and bipolar disorder. Specific learning disorders include, but are not limited to dyslexia and dyscalculia. Autism spectrum disorder (ASD) includes but, are not limited to Asperger's syndrome and Autistic Disorder. Motor disorders include, but are not limited to developmental coordination disorder and stereotypic movement disorder. Tic disorders include, but are not limited to Tourette's syndrome. Traumatic brain injury includes, but is not limited to congenital injuries such as those that cause cerebral palsy. Genetic disorders include, but are not limited to, fragile-X syndrome, Down syndrome, schizotypal disorder, and hypogonadotropic hypogonadal syndromes. Disorders associated with neurotoxicants include, but are not limited to, fetal alcohol spectrum disorder, Minamata disease caused by mercury, behavioral disorders including conduct disorder caused by heavy metals, such as lead, chromium and platinum, disorders caused by hydrocarbons such as dioxin, PBDEs and PCBs, disorders caused by medications and illegal drugs, such as cocaine and others. It should be appreciated that any disorder associated with TRN dysfunction is encompassed by aspects of the disclosure.

Aspects of the present disclosure provide methods for treating a psychiatric disorder using an effective amount of a group II metabotropic glutamate receptor modulator. As used herein, a “psychiatric disorder” refers to any disorder related to health conditions involving changes in emotion, thinking or behavior, or a combination thereof. Psychiatric disorders can be associated with distress and/or problems functioning in social, work or family activities. In some embodiments, the psychiatric disorder is not a schizophrenia disorder. In some embodiments, the psychiatric disorder as disclosed herein is not associated with any disorder or symptom associated with sleep spindle deficits.

Examples of psychiatric disorders include, but are not limited to, anxiety disorders such as generalized anxiety disorder, agoraphobia, social anxiety disorder, specific phobias, panic disorders, and separation anxiety disorder, trauma and stressor-related disorders such as acute stress disorder, adjustment disorders, post-traumatic stress disorder, and reactive attachment disorder, dissociative disorders such as dissociative amnesia, dissociative identity disorder, and depersonalization/derealization disorder, somatic symptom and related disorders such as somatic symptom disorder, illness anxiety disorder, conversion disorder, and factitious disorder, feeding and eating disorders such as anorexia nervosa, bulimia nervosa, rumination disorder, pica, binge-eating disorder, disruptive, impulse-control, and conduct disorders such as kleptomania, pyromania, intermittent explosive disorder, conduct disorder, and oppositional defiant disorder, depressive disorders such as disruptive mood dysregulation disorder, major depressive disorder, persistent depressive disorder (dysthymia), other or unspecified depressive disorder, premenstrual dysphoric disorder, substance/medication-induced depressive disorder, depressive disorder due to another medical condition, substance-related and addictive disorders such as alcohol-related disorders, cannabis-related disorders, inhalant-use disorder, stimulant use disorder, and tobacco use disorder, obsessive-compulsive and related disorders such as obsessive-compulsive disorder (OCD), body-dysmorphic disorder, hoarding disorder, trichotillomania (hair-pulling disorder), excoriation disorder (skin picking), substance/medication-induced obsessive-compulsive and related disorder, and obsessive-compulsive and related disorder due to another medical condition, obsessive-compulsive disorder such as obsessions and compulsions, personality disorders such as antisocial personality disorder, avoidant personality disorder, borderline personality disorder, dependent personality disorder, histrionic personality disorder, narcissistic personality disorder, obsessive-compulsive personality disorder, and paranoid personality disorder.

Aspects of the present disclosure provide methods for treating a sleep disorder using an effective amount of a group II metabotropic glutamate receptor modulator. As used herein, a “sleep disorder” refers to any disorder that involves disruption of the quality, timing and/or amount of sleep in a subject. Examples of sleep disorders include, but are not limited to, insomnia, restless legs syndrome (RLS), narcolepsy, circadian rhythm sleep disorders, shift work sleep disorder, delayed sleep phase disorder, jet lag, central sleep apnea, hypersomnia, parasomnia, rapid eye movement (REM) sleep behavior disorder, non-REM sleep arousal disorders, non-24-hour sleep-wake disorder, periodic limb movement disorder, sleep related hypoventilation, nightmare disorder, and hypersomnolence disorder. In some embodiments, the sleep disorder is not related to or caused by a schizophrenia disorder. In some embodiments, the sleep disorder as disclosed herein is not associated with any disorder or symptom associated with sleep spindle deficits.

Aspects of the disclosure relate to treating subjects having, suspected of having, or at risk of developing a neurodevelopmental disorder, a psychiatric disorder, or a sleep disorder. A subject to be treated by methods described herein may be a human subject or a non-human subject. Non-human subjects include, for example: non-human primates; farm animals, such as cows, horses, goats, sheep, and pigs; domestic animals, such as dogs and cats; and rodents.

In some embodiments, a subject does not have, is not suspected of having, or is not at risk of developing a schizophrenia disorder. In some embodiments, a subject does not have, is not suspected of having, or is not at risk of developing a disorder associated with sleep spindles.

A subject having a neurodevelopmental disorder, a psychiatric disorder, or a sleep disorder can be identified by routine medical examination, e.g., laboratory tests, neurological exams, physical exams, and/or brain imaging, as would be understood by one of ordinary skill in the art. In some embodiments, a subject having a neurodevelopmental disorder, a psychiatric disorder, or a sleep disorder has been diagnosed as having a neurodevelopmental disorder, a psychiatric disorder, or a sleep disorder. In other embodiments, a subject having a neurodevelopmental disorder, a psychiatric disorder, or a sleep disorder has not been diagnosed as having a neurodevelopmental disorder, a psychiatric disorder, or a sleep disorder.

A subject suspected of having a neurodevelopmental disorder, a psychiatric disorder, or a sleep disorder may be a subject that exhibits one or more symptoms of a neurodevelopmental disorder, a psychiatric disorder, or a sleep disorder, e.g., lack of facial expressions, inability to maintain eye contact, mood swings, irritable temper, memory and/or learning impairment, difficulty understanding and/or speaking, impulse control issues, and/or coordination issues. Other symptoms may include, but are not limited to, irritability, anxiety, lack of concentration, daytime fatigue, difficulty falling or staying asleep, excessive fears or worries, major changes in personality, eating habits, and/or sleeping patterns, feelings of guilt or worthlessness, and avoidance of social activities. In other embodiments, the subject has not exhibited any symptoms of a neurodevelopmental disorder, a psychiatric disorder, or a sleep disorder and/or has no history of neurodevelopmental disorders, psychiatric disorders, or sleep disorders. A subject at risk for developing a neurodevelopmental disorder, a psychiatric disorder, or a sleep disorder may be a subject having one or more of the risk factors for that disorder. For example, risk factors associated with a neurodevelopmental disorder, a psychiatric disorder, or a sleep disorder can include in some embodiments: (a) genetic factors; (b) age; (c) family history of a neurodevelopmental disorder, a psychiatric disorder, or a sleep disorder; and/or (d) exposure to certain toxins.

In some embodiments, the subject is a human subject having, suspected of having, or at risk for developing attention-deficit hyperactivity disorder (ADHD). In some embodiments, the subject is a human subject having, suspected of having, or at risk for developing an intellectual disability (ID). In some embodiments, the subject is a human subject having, suspected of having, or at risk for developing an autism spectrum disorders (ASD). In some embodiments, the subject is a human subject having, suspected of having, or at risk for developing a learning disorder. In some embodiments, the subject is a human subject having, suspected of having, or at risk for developing a motor disorder. In some embodiments, the subject is a human subject having, suspected of having, or at risk for developing a tic disorder. In some embodiments, the subject is a human subject having, suspected of having, or at risk for developing a traumatic brain injury. In some embodiments, the subject is a human subject having, suspected of having, or at risk for developing a communication, speech and/or language disorder. In some embodiments, the subject is a human subject having, suspected of having, or at risk for developing a genetic disorder. In some embodiments, the subject is a human subject having, suspected of having, or at risk for developing a disorder due to neurotoxicants.

In some embodiments, the subject is a human subject having, suspected of having, or at risk for developing insomnia. In some embodiments, the subject is a human subject having, suspected of having, or at risk for developing restless legs syndrome (RLS). In some embodiments, the subject is a human subject having, suspected of having, or at risk for developing narcolepsy. In some embodiments, the subject is a human subject having, suspected of having, or at risk for developing circadian rhythm sleep disorders. In some embodiments, the subject is a human subject having, suspected of having, or at risk for developing shift work sleep disorder. In some embodiments, the subject is a human subject having, suspected of having, or at risk for developing delayed sleep phase disorder. In some embodiments, the subject is a human subject having, suspected of having, or at risk for developing jet lag. In some embodiments, the subject is a human subject having, suspected of having, or at risk for developing central sleep apnea. In some embodiments, the subject is a human subject having, suspected of having, or at risk for developing hypersomnia. In some embodiments, the subject is a human subject having, suspected of having, or at risk for developing parasomnia. In some embodiments, the subject is a human subject having, suspected of having, or at risk for developing rapid eye movement (REM) sleep behavior disorder. In some embodiments, the subject is a human subject having, suspected of having, or at risk for developing non-REM sleep arousal disorders. In some embodiments, the subject is a human subject having, suspected of having, or at risk for developing non-24-hour sleep-wake disorder. In some embodiments, the subject is a human subject having, suspected of having, or at risk for developing periodic limb movement disorder. In some embodiments, the subject is a human subject having, suspected of having, or at risk for developing sleep related hypoventilation. In some embodiments, the subject is a human subject having, suspected of having, or at risk for developing nightmare disorder. In some embodiments, the subject is a human subject having, suspected of having, or at risk for developing hypersomnolence disorder. In some embodiments, the subject is a human subject having, suspected of having, or at risk for developing any symptom related to a sleep disorder.

In some embodiments, the subject is a human subject having, suspected of having, or at risk for developing an anxiety disorder. In some embodiments, the subject is a human subject having, suspected of having, or at risk for developing a trauma and stressor-related disorder. In some embodiments, the subject is a human subject having, suspected of having, or at risk for developing a dissociative disorder. In some embodiments, the subject is a human subject having, suspected of having, or at risk for developing a somatic symptom and related disorder. In some embodiments, the subject is a human subject having, suspected of having, or at risk for developing a feeding and/or eating disorder. In some embodiments, the subject is a human subject having, suspected of having, or at risk for developing a depressive disorder. In some embodiments, the subject is a human subject having, suspected of having, or at risk for developing a substance-related and/or addictive disorder. In some embodiments, the subject is a human subject having, suspected of having, or at risk for developing a substance/medication-induced depressive disorder. In some embodiments, the subject is a human subject having, suspected of having, or at risk for developing an obsessive-compulsive related disorder. In some embodiments, the subject is a human subject having, suspected of having, or at risk for developing a personality disorder. In some embodiments, the subject is a human subject having, suspected of having, or at risk for developing a conduct disorder. In some embodiments, the subject is a human subject having, suspected of having, or at risk for developing a depressive disorder due to another medical condition. In some embodiments, the subject is a human subject having, suspected of having, or at risk for developing a substance/medication-induced obsessive-compulsive related disorder. In some embodiments, the subject is a human subject having, suspected of having, or at risk for developing obsessive-compulsive disorder. In some embodiments, the subject is a human subject having, suspected of having, or at risk for developing any symptom related to a psychiatric disorder.

In some embodiments, the subject is a human subject exhibiting at least one symptom of attention-deficit hyperactivity disorder (ADHD). For example, the subject may be forgetful, easily distracted, impulsive, disorganized, or a combination thereof. In some embodiments, the subject has not exhibited any symptoms of ADHD and/or has no history of ADHD.

In some embodiments, the subject is a human subject exhibiting at least one symptom of an intellectual disability (ID). For example, the subject may have a deficit in an intellectual function including, but not limited to, language development, reasoning, problem solving, planning, abstract thinking, judgment, academic learning, and learning from experience. In some embodiments, the subject has not exhibited any symptoms of ID and/or has no history of ID.

In some embodiments, the subject is a human subject exhibiting at least one symptom of an autism spectrum disorders (ASD). For example, the subject may have exhibit difficulty with verbal and non-verbal communication, difficulty recognizing emotions, gauging personal space, repetitive body movements, and narrow interests. In some embodiments, the subject has not exhibited any symptoms of ASD and/or has no history of ASD. Examples of ASD include, but are not limited to, autistic disorder, Asperger's syndrome, pervasive developmental disorder not otherwise specified (PDD-NOS), pervasive developmental disorders, childhood disintegrative disorder, and high-functioning autism.

In some embodiments, the subject is a human subject exhibiting at least one symptom of anxiety disorders. In some embodiments, the subject is a human subject exhibiting at least one symptom of trauma and stressor-related disorders. In some embodiments, the subject is a human subject exhibiting at least one symptom of dissociative disorders. In some embodiments, the subject is a human subject exhibiting at least one symptom of somatic symptom and related disorders. In some embodiments, the subject is a human subject exhibiting at least one symptom of feeding and/or eating disorders. In some embodiments, the subject is a human subject exhibiting at least one symptom of conduct disorders. In some embodiments, the subject is a human subject exhibiting at least one symptom of depressive disorders. In some embodiments, the subject is a human subject exhibiting at least one symptom of substance-related and/or addictive disorders. In some embodiments, the subject is a human subject exhibiting at least one symptom of obsessive-compulsive related disorders. In some embodiments, the subject is a human subject exhibiting at least one symptom of substance/medication-induced obsessive-compulsive and related disorder. In some embodiments, the subject is a human subject exhibiting at least one symptom of obsessive-compulsive disorder. In some embodiments, the subject is a human subject exhibiting at least one symptom of personality disorders.

In some embodiments, the subject is a human subject exhibiting at least one symptom of insomnia. In some embodiments, the subject is a human subject exhibiting at least one symptom of restless legs syndrome (RLS). In some embodiments, the subject is a human subject exhibiting at least one symptom of narcolepsy. In some embodiments, the subject is a human subject exhibiting at least one symptom of circadian rhythm sleep disorders. In some embodiments, the subject is a human subject exhibiting at least one symptom of shift work sleep disorder. In some embodiments, the subject is a human subject exhibiting at least one symptom of delayed sleep phase disorder. In some embodiments, the subject is a human subject exhibiting at least one symptom of jet lag. In some embodiments, the subject is a human subject exhibiting at least one symptom of central sleep apnea. In some embodiments, the subject is a human subject exhibiting at least one symptom of hypersomnia. In some embodiments, the subject is a human subject exhibiting at least one symptom of parasomnia. In some embodiments, the subject is a human subject exhibiting at least one symptom of rapid eye movement (REM) sleep behavior disorder. In some embodiments, the subject is a human subject exhibiting at least one symptom of non-REM sleep arousal disorders. In some embodiments, the subject is a human subject exhibiting at least one symptom of non-24-hour sleep-wake disorder. In some embodiments, the subject is a human subject exhibiting at least one symptom of periodic limb movement disorder. In some embodiments, the subject is a human subject exhibiting at least one symptom of nightmare disorder. In some embodiments, the subject is a human subject exhibiting at least one symptom of hypersomnolence disorder.

Aspects of the disclosure relate to methods of improving sleep quality and/or increasing sleep duration in a subject using an effective amount of a group II metabotropic glutamate receptor modulator. In some embodiments, a subject is a healthy subject. In some embodiments, a subject does not have, is not suspected of having, and/or is not at risk of developing a neurodevelopmental disorder, a psychiatric disorder, or a sleep disorder. In other embodiments, a subject does have, is suspected of having, and/or is at risk of developing, a neurodevelopmental disorder, a psychiatric disorder, or a sleep disorder.

“An effective amount” of a group II metabotropic glutamate receptor modulator as used herein refers to the amount of a group II metabotropic glutamate receptor modulator that can confer a beneficial and/or therapeutic effect on a subject, either alone or in combination with one or more other active agents. In some aspects of the present disclosure, “an effective amount” of a group II metabotropic glutamate receptor modulator as used herein refers to the amount of a group II metabotropic glutamate receptor modulator that can improve sleep quality and/or increase sleep duration in a subject who has not been identified as having a disorder.

Effective amounts vary, as recognized by those skilled in the art, depending on the particular condition being treated, the severity of the condition, the individual patient parameters including age, physical condition, size, gender and weight, the duration of the treatment, the nature of concurrent therapy (if any), the specific route of administration and like factors within the knowledge and expertise of the health practitioner. These factors are well known to those of ordinary skill in the art and can be addressed without undue experimentation. In some embodiments, it is preferred that a maximum dose of the individual components or combinations thereof be used, that is, the highest safe dose according to sound medical judgment. It will be understood by those of ordinary skill in the art, however, that a patient may insist upon a lower dose or tolerable dose for medical reasons, psychological reasons or for any other reasons.

As used herein, the term “treating” a neurodevelopmental disorder, a psychiatric disorder, or a sleep disorder refers to the application or administration of one or more therapeutic agents to a subject who has a neurodevelopmental disorder, a psychiatric disorder, or a sleep disorder, a symptom of a neurodevelopmental disorder, a psychiatric disorder, or a sleep disorder, or a predisposition toward a neurodevelopmental disorder, a psychiatric disorder, or a sleep disorder with the purpose to cure, heal, alleviate, relieve, alter, remedy, ameliorate, improve, or affect the neurodevelopmental disorder, psychiatric disorder, or sleep disorder, at least one symptom of the neurodevelopmental disorder, psychiatric disorder, or sleep disorder or the predisposition toward the neurodevelopmental disorder, psychiatric disorder, or sleep disorder.

Alleviating a neurodevelopmental disorder, a psychiatric disorder, or a sleep disorder can include delaying the development or progression of the neurodevelopmental disorder, psychiatric disorder, or sleep disorder or reducing the severity of the neurodevelopmental disorder, psychiatric disorder, or sleep disorder. Alleviating the neurodevelopmental disorder, psychiatric disorder, or sleep disorder does not necessarily require curative results. As used herein, “delaying” the development of a neurodevelopmental disorder (such as ADHD), a psychiatric disorder, or a sleep disorder means to defer, hinder, slow, retard, stabilize, and/or postpone progression of the neurodevelopmental disorder, psychiatric disorder, or sleep disorder. This delay can be of varying lengths of time, depending on the history of the disease and/or the individual being treated. A method that “delays” or alleviates the development of a neurodevelopmental disorder, a psychiatric disorder, or a sleep disorder, or delays the onset of the neurodevelopmental disorder, psychiatric disorder, or sleep disorder, can be a method that reduces probability of developing one or more symptoms of the neurodevelopmental disorder, psychiatric disorder, or sleep disorder in a given time frame and/or reduces the extent of the symptoms in a given time frame, when compared to a control. Such comparisons may, in some embodiments, be based on clinical studies, using a number of subjects sufficient to give a statistically significant result.

“Development” or “progression” of a neurodevelopmental disorder, a psychiatric disorder, or a sleep disorder refers to initial manifestations and/or ensuing progression of the neurodevelopmental disorder, psychiatric disorder, or sleep disorder. Development or progression of a neurodevelopmental disorder, a psychiatric disorder, or a sleep disorder can in some instances be detectable and assessed using standard clinical techniques known in the art. However, in other instances, development or progression may be undetectable.

### Identification of Subjects Having Neurodevelopmental Disorders, Psychiatric Disorders, or Sleep Disorders

Methods described herein encompass detecting a neurodevelopmental disorder, a psychiatric disorder, or a sleep disorder. In some embodiments, detecting a neurodevelopmental disorder, a psychiatric disorder, or a sleep disorder comprises detecting a TRN dysfunction. In some embodiments, a TRN dysfunction comprises a dysfunction, relative to a control, in any characteristic related to a TRN, of at least 5%, 10%, 20%, 30%, 40%, 50%, 60%, 70%, 80%, 90%, 100%, or at least 2-fold, at least 5-fold, at least 10-fold, at least 20-fold, at least 50-fold, or at least 100-fold. Examples of a characteristic related to a TRN include, but are not limited to, TRN function (e.g., repetitive burst firings of TRN neurons), TRN genetics (e.g., gene expression and/or mutation), TRN neural circuitry, and behavior changes. In some embodiments, a TRN dysfunction comprises a dysfunction in at least one characteristic related to a TRN. In some embodiments, a TRN dysfunction comprises a dysfunction in at least two, at least three, at least four, at least five, at least six, at least seven, at least eight, at least nine, or at least ten characteristics related to a TRN. In some embodiments, a TRN dysfunction comprises a dysfunction in more than one characteristic related to a TRN.

In some embodiments, detecting a neurodevelopmental disorder comprises detecting a mutation in the PTCHD1 gene. The X-linked PTCHD1 gene is predicted to encode a twelve-pass transmembrane protein with a sterol-sensing domain (SSD), and therefore it is classified as a member of the Patched family. Prenatally, PTCHD1 is expressed in the developing cerebellum and diencephalon. Ptchd1 mRNA is confined to the thalamic reticular nucleus (TRN) at birth, and thereafter it is expressed in the TRN, striatum, cortex, and cerebellum. Patched family members PTCHD2 and PTCHD3 showed no TRN expression. The amino acid sequence of the human PTCHD1 protein is provided, for example, in RefSeq No. NP_775766.2 and UniProt No. Q96NR3-1. The nucleic acid sequence of the human PTCHD1 gene is provided, for example, in RefSeq No. NM_173495.2.

Aspects of the disclosure relate to identifying subjects having a neurodevelopmental disorder based, at least in part, on whether the subject has a mutation in the PTCHD1 gene. In some embodiments the mutation in the PTCHD1 gene is a loss-of-function mutation. As used herein, a “loss-of-function” mutation refers to any mutation that reduces the function of the gene product. In some embodiments, a mutation in the PTCHD1 gene comprises an insertion, deletion, and/or substitution in the PTCHD1 gene or PTCHD1 protein product thereof.

Detecting a mutation in the PTCHD1 gene can comprise detecting a mutation in the nucleic acid encoding the PICHD1 gene and/or detecting a mutation in the PTCHD1 protein, and can be achieved by any means known in the art. For example, a mutation in the nucleic acid (DNA or RNA) encoding the PTCHD1 gene could be detected by, e.g., a sequencing assay, a polymerase chain reaction (PCR) assay, and/or a nucleic acid microarray assay. A mutation in the PTCHD1 protein could be detected by, e.g., an immunohistochemical assay, an immunoblotting assay, a flow cytometry assay, and/or a mass spectrometry assay.

In some embodiments, detecting a psychiatric disorder comprises detecting any genetic alteration associated with a psychiatric disorder. In some embodiments, detecting a psychiatric disorder does not comprise detecting any genetic alteration associated with a sleep spindle deficit. In some embodiments, detecting a sleep disorder comprises detecting any genetic alteration associated with a sleep disorder. For example, a genetic alteration associated with a sleep disorder may comprise genetic alterations associated with fatal familial insomnia, familial advanced sleep-phase syndrome, chronic primary insomnia, and narcolepsy with cataplexy. In some embodiments, detecting a sleep disorder does not comprise detecting any genetic alteration associated with a sleep spindle deficit. In some embodiments, detecting a sleep disorder comprises reviewing sleep log, sleep inventory, blood tests, and/or sleep study.

### Group II Metabotropic Glutamate Receptor Modulators

The metabotropic glutamate receptors (mGluRs) are a family G-protein-coupled receptors that participate in the modulation of synaptic transmission and neuronal excitability throughout the central nervous system. The mGluRs bind glutamate within a large extracellular domain and transmit signals through the receptor protein to intracellular signaling partners. Genes encoding eight mGluR subtypes have been identified, many with multiple splice variants that are differentially expressed in distinct cell types throughout the CNS. mGluRs are subclassified into three groups based on sequence homology, G-protein coupling, and ligand selectivity. Group I includes mGluRs 1 and 5, Group II includes mGluRs 2 and 3, and Group III includes mGluRs, 4, 6, 7, and 8.

Aspects of the disclosure relate to treating a neurodevelopmental disorder, a psychiatric disorder, or a sleep disorder with group II metabotropic glutamate receptor modulators. In some embodiments, the group II metabotropic glutamate receptor modulator blocks or inhibits a biological response by binding to a group II metabotropic glutamate receptor (e.g., a group II metabotropic glutamate receptor antagonist). In other embodiments, the group II metabotropic glutamate receptor modulator binds to a group II metabotropic glutamate receptor and activates the receptor to produce a biological response (e.g., a group II metabotropic glutamate receptor agonist). In other embodiments, the group II metabotropic glutamate receptor modulator modulates a biological response of a group II metabotropic glutamate receptor allosterically by binding to a site different than the ligand binding site (e.g., a group II metabotropic glutamate receptor positive allosteric modulator (PAM) or a group II metabotropic glutamate receptor negative allosteric modulator (NAM)).

A group II metabotropic glutamate receptor modulator, as used herein, refers to any ligand that modulates (e.g., increases or decreases) a biological response of a group II metabotropic glutamate receptor. Group II metabotropic glutamate receptor modulators include group II metabotropic glutamate receptor 2 (mGlu₂) and group II metabotropic glutamate receptor 3 (mGlu₃) modulators.

In some embodiments, a group II metabotropic glutamate receptor modulator used in the methods described herein increases a biological response of a group II metabotropic glutamate receptor by at least 5%, 10⁰%, 20%, 30%, 40%, 50%, 60%, 70%, 80%, 90%, 100%, or by at least 2-fold, at least 5-fold, at least 10-fold, at least 20-fold, at least 50-fold, at least 100-fold, or at least 1000-fold. In some embodiments, a group II metabotropic glutamate receptor modulator used in the methods described herein decreases a biological response of a group II metabotropic glutamate receptor by at least 5%, 10%, 20%, 30⁰%, 40%, 50%, 60%, 70%, 80%, 900%, 100%, or by at least 2-fold, at least 5-fold, at least 10-fold, at least 20-fold, at least 50-fold, at least 100-fold, or at least 1000-fold.

Examples of group II metabotropic glutamate receptor modulators include, but are not limited to, group II metabotropic glutamate receptor agonists, group II metabotropic glutamate receptor antagonists, group II metabotropic glutamate receptor negative allosteric regulators (NAMs), and group II metabotropic glutamate receptor positive allosteric regulators (PAMs).

In some embodiments, the group II metabotropic glutamate receptor modulator interacts with both mGlu₂ and mGlu₃. In certain embodiments, the group II metabotropic glutamate receptor modulator interacts differently with mGlu₂ and mGlu₃. For example, the group II metabotropic glutamate receptor modulator may be a mGlu₂ agonist and a mGlu₃ antagonist (e.g., LY395756). In other embodiments, the group II metabotropic glutamate receptor modulator may be a mGlu₂ antagonist and a mGlu₃ agonist.

In some embodiments, the group II metabotropic glutamate receptor modulator specifically interacts with mGlu₂ or mGlu₃. Specific interaction of a ligand with a receptor, such as mGlu₂ or mGlu₃, is well-understood in the art, and methods to determine such specific interactions are also well-known in the art. A group II metabotropic glutamate receptor modulator is said to exhibit specific interaction with a receptor, such as mGlu₂ or mGlu₃, if it reacts or associates more frequently, more rapidly, with greater duration, and/or with greater affinity with a receptor, such as mGlu₂ or mGlu₃, than it does with another receptor. It should also be understood that a ligand that specifically interacts with mGlu₂ or mGlu₃ may or may not specifically or preferentially interact with another receptor (e.g., a group I mGlu receptor). As such, specific interaction or preferential interaction does not necessarily require (although it can include) exclusive binding or interaction.

In some embodiments, the group II metabotropic glutamate receptor modulator is a mGlu₃-specific modulator. Examples of mGlu₃-specific modulators include, but are not limited to, mGlu₃ agonists, mGlu₃ antagonists, mGlu₃ NAMs, and mGlu₃ PAMs.

In some embodiments, mGluR₃ is selectively enriched in the TRN. In some embodiments, the augmentation of mGluR₃ activity by an mGluR₃ PAM corrects the reduced rebound bursting of TRN neurons. In some embodiments, mGluR₃-specific modulators can correct or improve sleep disruption. In some embodiments, the mGluR₃-specific modulator is a mGluR₃ PAM. In some embodiments, the mGluR₃-specific modulator is a mGluR₃ agonist. In some embodiments, the mGluR₃ PAM is Mavalon 63. In some embodiments, the mGluR₃ PAM can be any positive allosteric modulator as disclosed herein. In some embodiments, an mGluR_{2/3} agonist can correct or improve sensory filtering deficits. In some embodiments, the mGluR_{2/3} agonist is LY379268. In some embodiments, the mGluR_{2/3} agonist can be any mGluR_{2/3} agonist suitable for the methods as disclosed herein.

In some embodiments, the group II metabotropic glutamate receptor modulator is a mGlu₂-specific modulator. Examples of mGlu₂-specific modulators include, but are not limited to, mGlu₂ agonists, mGlu₂ antagonists, mGlu₂ NAMs, and mGlu₂ PAMs.

In some embodiments, the group II metabotropic glutamate receptor modulator is an mGlu₂ and mGlu₃ agonist. In some embodiments, the group II metabotropic glutamate receptor agonist is selected from the group consisting of LY354740, MGS0028, LY379268, LY2934747, LY2969822, LY404040, LY404039, and LY2140023.

In some embodiments, the group II metabotropic glutamate receptor modulator is an mGlu₂ and mGlu₃ antagonist. In some embodiments, the group II metabotropic glutamate receptor antagonist is selected from the group consisting of LY341495, LY3020371, HYDIA, MGS0039, and CECXG.

In some embodiments, the group II metabotropic glutamate receptor modulator is an mGlu₂ and mGlu₃ NAM. In some embodiments, the mGlu₂ and mGlu₃ NAM is RO4491533 or MNI-137. In some embodiments, the mGlu₂ and mGlu₃ NAM is RO4491533. In some embodiments, the mGlu₂ and mGlu₃ NAM is MNI-137.

In some embodiments, the group II metabotropic glutamate receptor modulator is a mGlu₂ PAM. In some embodiments, the mGlu₂ PAM is selected from the group consisting of AZD8529, ADX-71149, JNJ-42153605, JNJ-40068782, GSK1331258, SAR218645, TASP0433864, LY487379, and BINA.

In some embodiments, the group II metabotropic glutamate receptor modulator is a mGlu₂ NAM. In some embodiments, the mGlu₂ NAM is VU6001966.

In some embodiments, the group II metabotropic glutamate receptor modulator is a mGlu₂ agonist. In some embodiments, the mGlu₂ agonist is LY2812223 or LY2979165. In some embodiments, the mGlu₂ receptor agonist is LY2812223. In some embodiments, the mGlu₂ receptor agonist is LY2979165.

In some embodiments, the group II metabotropic glutamate receptor modulator is a mGlu₃ agonist. In some embodiments, the mGlu₃ agonist is LY2794193.

In some embodiments, the group II metabotropic glutamate receptor modulator is a mGlu₃ PAM. In some embodiments, the mGlu₃ PAM is selected from the group consisting of DT011088, Mav63 PAM, and Mav207 PAM.

In some embodiments, the group H metabotropic glutamate receptor modulator is a mGlu₃ NAM. In some embodiments, the mGlu₃ NAM is selected from the group consisting of VU0650786, ML337, and LY2389575. In some embodiments, the group II metabotropic glutamate receptor modulator is a group II metabotropic glutamate receptor modulator disclosed in, and incorporated by reference from, US2015361081 or WO2016130652.

Examples of group II metabotropic glutamate receptor modulators and their structures are provided in Table 1.

TABLE 1
Non-limiting examples of group II metabotropic glutamate receptor modulators

| Name | Chemical Name | Chemical Structure | Function | Reference |
|---|---|---|---|---|
| LY2794193 | (1S,2S,4S,5R,6S)- 2-amino-4-[(3- methoxybenzoyl) amino]bicyclo[3.1.0] hexane-2,6- dicarboxylic acid | 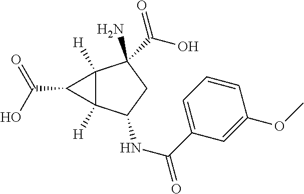 [SMILES: COc1cccc(C(=O)N[C@H]2C[C@@](N)(C(=O)O)[C@@H]3[C@@H](C(=O)O)[C@@H]32)c1] | mGlu₃ Agonist | Journal of Medicinal Chemistry (2018), 61(6), 2303-2328 |
| DT011088 | Not reported | Not reported | mGlu₃ | Domain |
| | | | PAM | Therapeutics |
| Mavalon 63 (Mav63) | 5-Methyl-9-(5- fluoropyridin-2- yl)-spiro [benzo[f]pyrrolo[1,2- a][1,4]diazepine-6,1′- cyclopropan]- 4(5H)-one | 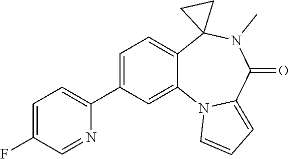 [SMILES: CN1C(=O)c2cccn2-c2cc(-c3ccc(F)cn3)ccc2C12CC2] | mGlu₃ PAM | WO20170 81483A1 WO20182 06820A1 |
| Mavalon 207 (Mav207) | 2-Methoxymethyl- 5-methyl-9-(6- fluoro-pyridin-3- yl)-spiro[benzo [f]pyrazolo[1,5- a][1,4]diazepine- 6,1′-cyclopropan]- 4(5H)-one | 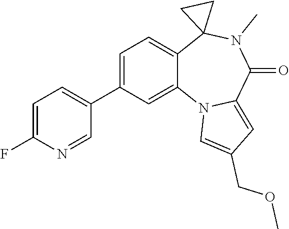 [SMILES: COCc1cc2n(c1)-c1cc(-c3ccc(F)nc3)ccc1C1(CC1)N(C)C2=O] | mGlu₃ PAM | WO20170 81483A1 WO20182 06820A1 |
| Merck example 1 | 1-isopropyl-3- morpholin-4-yl- 5,6,7,8-tetrahydro- isoquinoline- 4-carbonitrile | 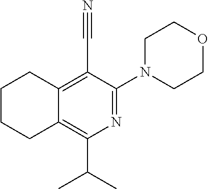 [SMILES: CC(C)c1nc(N2CCOCC2)c(C#N)c2c1CCCC2] | mGlu₃ PAM | WO20141 17919A1 |
| VU0650786 | (R)-2-(((5- chloropyridin-2- yl)oxy)methyl)-5- (2-fluoropyridin-3- yl)-7-methyl-6,7- dihydropyrazolo[1,5- a]pyrazin-4(5H)-one | 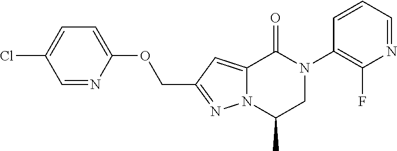 [SMILES: C[C@@H]1CN(c2cccnc2F)C(=O)c2cc(COc3ccc(Cl)cn3)nn21] | mGlu₃ NAM | Journal of Medicinal Chemistry (2015), 58(18), 7485-7500 |
| ML337 | (R)-(2-fluoro-4-((- 4-methoxy- phenyl)ethynyl) phenyl) (3- hydroxypiperidin- 1-yl)methanone | 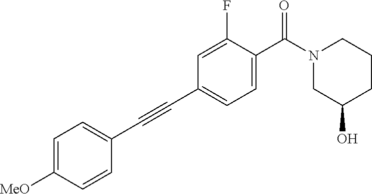 [SMILES: COc1ccc(C#Cc2ccc(C(=O)N3CCC[C@@H](O)C3)c(F)c2)cc1] | mGlu₃ NAM | Journal of Medicinal Chemistry (2013), 56(12), 5208-5212 |
| LY2389575 | (3S)-N-(2,4- Dichlorobenzyl)- 1-(5- bromopyrimidin- 2-yl)pyrrolidinyl- 3-amine | 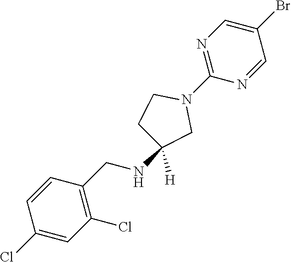 [SMILES: Clc1ccc(CN[C@H]2CCN(c3ncc(Br)cn3)C2)c(Cl)c1] | mGlu₃ NAM | WO20060 44454A1 |
| US201536 1081 Compound 37 | 2-(((4- (difluoromethoxy)-2- fluorophenyl)amino) methyl)-5-(4- fluorophenyl)-6,7- dihydropyrazolo[1,5- a]pyrazin-4(5H)-one | 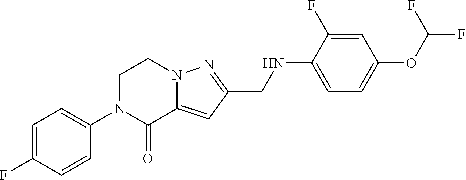 [SMILES: O=C1c2cc(CNc3ccc(OC(F)F)cc3F)nn2CCN1c1ccc(F)cc1] | mGlu₃ NAM | US201536 1081 |
| WO201613 0652 example 1 | 1-(4- Fluorophenyl)-4- hydroxypyridin- 2(1H)-one | 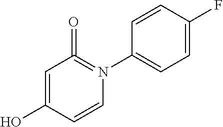 | mGlu₃ NAM | WO20161 30652 |
| LY2812223 | (1R,2S,4R,5R,6R)- 2-amino-4-(1H- 1,2,4-triazol-3- ylsulfanyl)bicyclo [3.1.0]hexane-2,6- dicarboxylic acid | 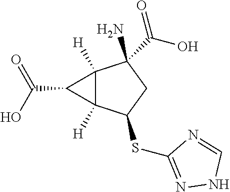 [SMILES: N[C@@]1(C(=O)O)C[C@@H](Sc2nc[nH]n2)[C@H]2[C@H](C(=O)O)[C@H]21] | mGlu₂ Agonist | US201101 52334A1 |
| LY2979165 | (1S,2R,4S,5S,6S)- 4-((4H-1,2,4- triazol-3-yl)thio)- 2-((R)-2- aminopropanamido) bicyclo[3.1.0] hexane-2,6- dicarboxylic acid | 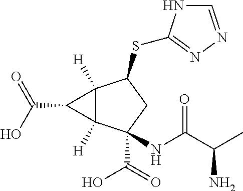 [SMILES: C[C@@H](N)C(=O)N[C@]1(C(=O)O)C[C@H](Sc2nnc[nH]2)[C@@H]2[C@@H](C(=O)O)[C@@H]21] | mGlu₂ Agonist | US201101 52334A1 |
| AZD8529 | 1H-Isoindol-1-one, 2,3-dihydro-7- methyl-5-[3-(1- piperazinylmethyl)- 1,2,4-oxadiazol- 5-yl]-2-[[4- (trifluoromethoxy) phenyl]methyl]- | 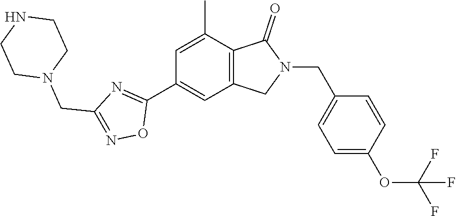 [SMILES: Cc1cc(-c2nc(CN3CCNCC3)no2)cc2c1C(=O)N(Cc1ccc(OC(F)(F)F)cc1)C2] | mGlu₂ PAM | US837794 0B2 and WO20081 50233A1 |
| ADX-71149 | 1-(4-Chloro-2- fluorobenzyl)-5- (4- methoxyphenyl)- 2(1H)-pyridinone | 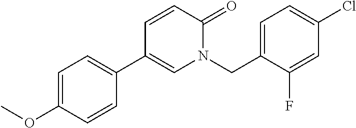 [SMILES: COc1ccc(-c2ccc(=O)n(Cc3ccc(Cl)cc3F)c2)cc1] | mGlu₂ PAM | WO20090 33704A1 |
| JNJ-42153605 | 3- (cyclopropylmethyl)- 7-(4- phenylpiperidin-1- yl)-8- (trifluoromethyl)- [1,2,4]triazolo[4,3- a]pyridine | 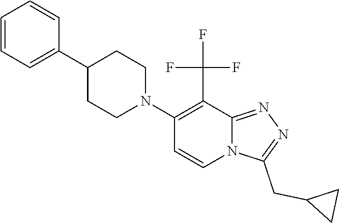 [SMILES: FC(F)(F)c1c(N2CCC(c3ccccc3)CC2)ccn2c(CC3CC3)nnc12] | mGlu₂ PAM | WO20101 30424A1 |
| JNJ-40068782 | 3-Cyano-1- cyclopropylmethyl- 4-(4-phenyl- piperidin-1-yl)- pyridine-2(1H)- one | 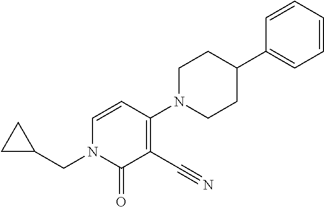 [SMILES: N#Cc1c(N2CCC(c3ccccc3)CC2)ccn(CC2CC2)c1=O] | mGlu₂ PAM | WO20081 07479A1 and WO20071 04783A2 |
| GSK1331258 | 2-[[4-[3-chloro-5- (trifluoromethyl) pyridin-2- yl]piperazin-1- yl]methyl]-1-methyl- benzimidazole | 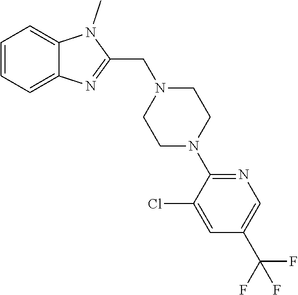 [SMILES: Cn1c(CN2CCN(c3ncc(C(F)(F)F)cc3Cl)CC2)nc2ccccc21] | mGlu₂ PAM | Bioorganic & Medicinal Chemistry Letters (2010), 20(2), 759-762 |
| SAR218645 | ((S)-2-(1,1-dimethyl- indan-5-yloxymethyl)- 2,3-dihydro- oxazolo[3,2- a]pyrimidin-7-one) | 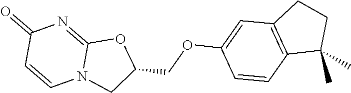 | mGlu₂ PAM | WO20110 34830A1 |
| TASP0433864 | [(2S)-2-[(4-tert- butylphenoxy)methyl]- 5-methyl-2,3- dihydroimidazo[2,1- b][1,3]oxazole- 6-carboxamide] | 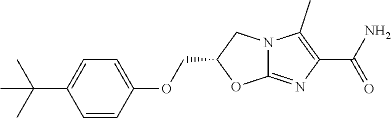 [SMILES: Cc1c(C(N)=O)nc2n1C[C@@H](COc1ccc(C(C)(C)C)cc1)O2] | mGlu₂ PAM | WO20130 62079A1 |
| LY487379 | 2,2,2-Trifluoro-N- [4-(2- methoxyphenoxy) phenyl]-N-(3- pyridinylmethyl) ethanesulfonamide | 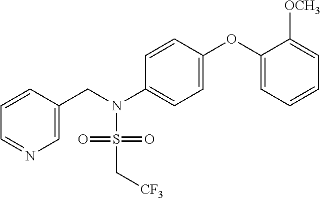 [SMILES: COc1ccccc1Oc1ccc(N(Cc2cccnc2)S(=O)(=O)CC(F)(F)F)cc1] | mGlu₂ PAM | WO20010 56990A2 |
| BINA | 3′-[[(2- Cyclopentyl-2,3- dihydro-6,7- dimethyl-1-oxo- 1H-inden-5- yl)oxy]methyl]- [1,1′-biphenyl]-4- carboxylic acid | 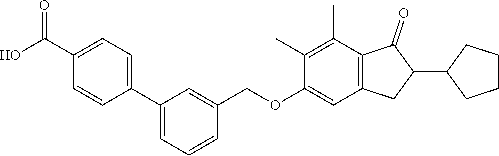 [SMILES: Cc1c(OCc2cccc(-c3ccc(C(=O)O)cc3)c2)cc2c(c1C)C(=O)C(C1CCCC1)C2] | mGlu₂ PAM | WO20060 15158A1 |
| VU6001966 | 4-(4- fluorophenyl)-5- ((1-methyl-1H- pyrazol-3- yl)methoxy) picolinamide | 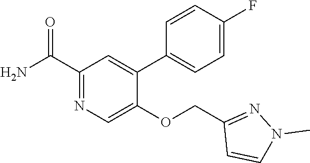 [SMILES: Cn1ccc(COc2cnc(C(N)=O)cc2-c2ccc(F)cc2)n1] | mGlu₂ NAM | WO20161 49324A1 |
| LY354740 | (1S,2S,5R,6S)-2- aminobicyclo[3.1.0] hexane-2,6- dicarboxylic acid | 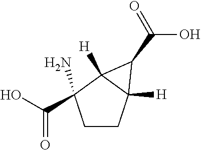 [SMILES: N[C@@]1(C(=O)O)CC[C@H]2[C@H](C(=O)O)[C@H]21] | mGlu₂ and mGlu₃ Agonist | EP696577A1 and U.S. Pat. No. 5,882,671 |
| MGS0028 | (1R,2S,5S,6S)-2- amino-6-fluoro-4- oxobicyclo[3.1.0] hexane-2,6- dicarboxylic acid | 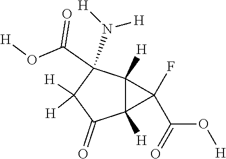 [SMILES: N[C@@]1(C(=O)O)CC(=O)[C@@H]2[C@H]1C2(F)C(=O)O] | mGlu₂ and mGlu₃ Agonist | WO20000 12464A1 |
| LY379268 | (1R,4R,5S,6R)-4- Amino-2- oxabicyclo[3.1.0] hexane-4,6- dicarboxylic acid | 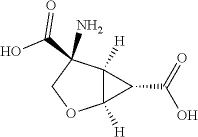 [SMILES: N[C@@]1(C(=O)O)CO[C@@H]2[C@@H](C(=O)O)[C@@H]21] | mGlu₂ and mGlu₃ Agonist | U.S. Pat. No. 5,688,826 and EP774461 |
| LY2934744 | (1R,4S,5S,6S)-4- aminospiro[bicyclo [3.1.0]hexane- 2,1′- cyclopropane]-4,6- dicarboxylic acid | 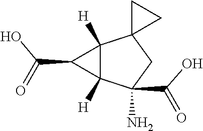 [SMILES: N[C@@]1(C(=O)O)CC2(CC2)[C@H]2[C@H](C(=O)O)[C@H]21] | mGlu₂ and mGlu₃ Agonist | US201301 97079A1 |
| LY2969822 | (1R,4S,5S,6S)-4- ((S)-2- aminopropanamido) spiro[bicyclo[3.1.0] hexane-2,1′- cyclopropane]-4,6- dicarboxylic acid | 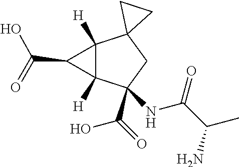 [SMILES: C[C@H](N)C(=O)N[C@@]1(C(=O)O)CC2(CC2)[C@H]2[C@H](C(=O)O)[C@H]21] | mGlu₂ and mGlu₃ Agonist | US201301 97079A1 |
| LY404040 | (1R,2R,4S,5S,6S)- 4-amino-2- hydroxy-214- thiabicyclo[3.1.0] hexane-4,6- dicarboxylic acid | 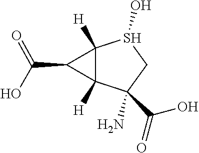 | mGlu₂ and mGlu₃ Agonist | Journal of Medicinal Chemistry (2007), 50(2), 233-240 |
| LY404039 (pomaglumetad) | (1R,4S,5S,6S)-4- Amino-2- thiabicyclo[3.1.0] hexane-4,6- dicarboxylic acid 2,2-dioxide | 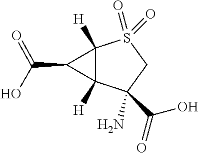 [SMILES: N[C@@]1(C(=O)O)CS(=O)(=O)[C@H]2[C@H](C(=O)O)[C@H]21] | mGlu₂ and mGlu₃ Agonist | WO20031 04217A2 |
| LY2140023 (pomaglumetad methionil) | (1R,4S,5S,6S)-4- [[(2S)-2-amino-4- methylsulfanyl- butanoyl]amino]- 2,2-dioxo-2λ⁶- thiabicyclo[3.1.0] hexane-4,6- dicarboxylic acid | 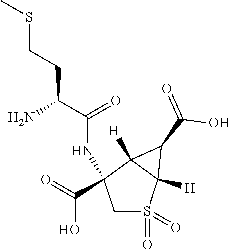 | mGlu₂ and mGlu₃ Agonist | WO20031 04217A2 US201102 37602A1 |
| LY341495 | (2S)-2-Amino-2- [(1S,2S)-2- carboxycycloprop- 1-yl]-3-(xanth-9- yl)propanoic acid | 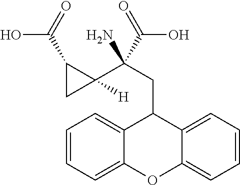 [SMILES: N[C@](CC1c2ccccc2Oc2ccccc21)(C(=O)O)[C@H]1C[C@@H]1C(=O)O] | mGlu₂ and mGlu₃ Ant- agonist | U.S. Pat. No. 5,717,109 |
| LY3020371 | (1S,2R,3S,4S,5R,6R)- 2-amino-3- [(3,4-difluorophenyl) sulfanylmethyl]-4- hydroxy- bicyclo[3.1.0] hexane-2,6- dicarboxylic acid | 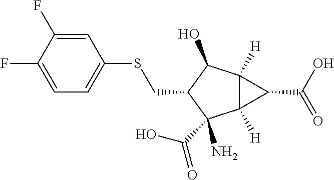 [SMILES: N[C@]1(C(=O)O)[C@@H]2[C@@H](C(=O)O)[C@@H]2[C@H](O)[C@H]1CSc1ccc(F)c(F)c1] | mGlu₂ and mGlu₃ Ant- agonist | WO20120 68067A1 |
| HYDIA | (1S,2R,3R,5R,6S)- 2-amino-3- hydroxy-bicyclo [3.1.0]hexane-2,6- dicarboxylic acid | 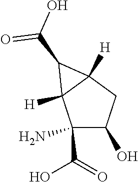 [SMILES: N[C@]1(C(=O)O)[C@H]2[C@@H](C[C@H]1O)[C@@H]2C(=O)O] | mGlu₂ and mGlu₃ Ant- agonist | DE199416 75A1 |
| MGS0039 | (1R,2R,3R,5R,6R)- 2-amino-3-[(3,4- dichlorophenyl) rnethoxy]-6- fluorobicyclo[3.1.0] hexane-2,6- dicarboxylic acid | 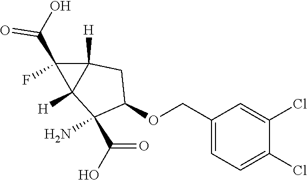 [SMILES: N[C@]1(C(=O)O)[C@H]2[C@@H](C[C@H]1OCc1ccc(Cl)c(Cl)c1)[C@]2(F)C(=O)O] | mGlu₂ and mGlu₃ Ant- agonist | WO20030 61698A1 |
| LY341495 (CECXG) | (1S,2S,3S)-2-[1- Amino-1-carboxy- 2-(9H-xanthen-9- yl)-ethyl]-3-ethyl- cyclopropane- carboxylic acid | 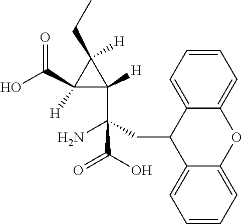 [SMILES: CC[C@@H]1[C@H](C(=O)O)[C@H]1[C@@](N)(CC1c2ccccc2Oc2ccccc21)C(=O)O] | mGlu₂ and mGlu₃ Ant- agonist | Bioorganic & Medicinal Chemistry Letters (1998), 8(20), 2849-2854 |
| RO4491533 | 4-[3-(2,6- dimethylpyridin-4- yl)phenyl]-7- methyl-8- (trifluoromethyl)- 1,3-dihydro-1,5- benzodiazepin-2- one | 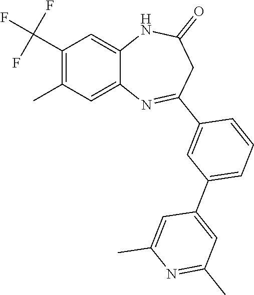 | mGlu₂ and mGlu₃ NAM | WO20030 66623A1 |
| MNI-137 | 4-(8-Bromo-2,3- dihydro-2-oxo-1H- 1,5-benzodiazepin- 4-yl)-2- pyridinecarbonitrile | 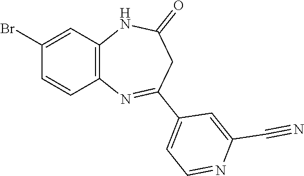 [SMILES: N#Cc1cc(C2=Nc3ccc(Br)cc3NC(=O)C2)ccn1] | mGlu₂ and mGlu₃ NAM | WO20151 91630A1 |
| LY395756 | (1S,2S,4R,5R,6S)- rel-2-Amino-4- methylbicyclo[3.1.0] hexane-2,6- dicarboxylic acid | 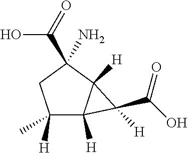 | mGlu₂ Agonist/ mGlu₃ Ant- agonist | EP774454 A1 |

A group II metabotropic glutamate receptor modulator can be administered one or more times to a subject. A group II metabotropic glutamate receptor modulator can also be administered as part of a combination therapy. In some embodiments, a subject receiving a group II metabotropic glutamate receptor modulator can also be administered an additional therapeutic agent.

An additional therapeutic agent can be a stimulant. Examples of a stimulant include, but are not limited to, amphetamine, methylphenidate, and lisdexamfetamine dimesylate. An additional therapeutic agent can be a non-stimulant, e.g., atomoxetine hydrochloride.

An additional therapeutic agent can be an antipsychotic. Examples of antipsychotics include, but are not limited to, aripiprazole, asenapine, brexpiprazole, cariprazine, clozapine, iloperidone, lurasidone, olanzapine, paliperidone, quetiapine, risperidone, ziprasidone, chlorpromazine, fluphenazine, haloperidol, and perphenazine.

An additional therapeutic agent can be a selective serotonin reuptake inhibitor (SSRI). Examples of SSRIs include, but are not limited to, citalopram, escitalopram, fluoxetine, fluvoxamine, fluvoxamine, paroxetine, paroxetine, sertraline.

An additional therapeutic agent can be a serotonin-norepinephrine reuptake inhibitor (SNRI). Examples of SNRIs include, but are not limited to, desvenlafaxine, duloxetine, venlafaxine, venlafaxine, milnacipran, and levomilnacipran.

An additional therapeutic agent can be a tricyclic antidepressant (TCA). Examples of TCAs include, but are not limited to, amitriptyline, desipramine, doxepine, imipramine, nortriptyline, amoxapine, clomipramine, maprotiline, trimipramine, and protriptyline.

An additional therapeutic agent can be a monoamine oxidase inhibitor (MAOI). Examples of MAOIs include, but are not limited to, phenelzine, selegiline, and tranylcypromine.

An additional therapeutic agent can be a benzodiazepine. Examples of benzodiazepines include, but are not limited to, alprazolam, clonazepam, diazepam, and lorazepam.

For intellectual disability (ID), intellectual and developmental disability (IDD) or mental retardation, an additional therapeutic agent can be metformin, memantine, flumazenil, or meclofenoxate. In some embodiments, an additional therapeutic agent for intellectual disability (ID), intellectual and developmental disability (IDD) or mental retardation can be in combination with Risperidone, Carbamazepine, Sodium Valproate, Lamotrigine, Lithium Carbonate, Methylphenidate, Procyclidine, Ferrous Fumarate+Vitamins+Lactulose+cod liver oil+various skin ointments, Clobazam+Lorazepam, Rectal diazepam+buccal midazolam.

For specific learning disorders, an additional therapeutic agent can be omega-3 fatty acids+inositol, n-acetylcysteine, intranasal oxytocin, memantine hydrochloride, lovastatin, BPN14770, dronabinol, THC, 18F-AV-1451, ketamine, midazolam, d-cycloserine, rivaroxaban, acetylsalicylic acid, metformin amisulpride, bromocriptine, or acetazolamide.

For autism spectrum disorders, an additional therapeutic agent can be antipsychotics including risperidone, aripiprazole, ziprasidone, SSRIs including fluoxetine, citalopram, escitalopram, stimulants including methylphenidate, or alpha-2-adrenergic agonists including clonidine, and guanfacine.

For motor disorders, an additional therapeutic agent can be propranolol, beta-blockers, primidone, clonazepam, diazepam, lorazepam, or alprazolam.

For tic disorders, an additional therapeutic agent can be pimozide, haloperidol, aripiprazole, dopamine antagonists, aripiprazole, antipsychotics including clonidine, risperidone, olanzapine, ziprasidone, haloperidol, fluphenazine, pimozide, tetrabenazine, guanfacine, skeletal muscle relaxants including baclofen, benzodiazepines including clonazepam, or neuromuscular blockers including onabotulinumtoxinA.

For traumatic brain injury, an additional therapeutic agent can be amantadine, cyclosporine A, donepezil, glyburide, lithium, methylphenidate, minocycline, progesterone, rivastigmine, or simvastatin.

For fragile-X syndrome, an additional therapeutic agent can be lithium, fenobam, γ-aminobutyric acid agonists, or metformin.

For Down syndrome, an additional therapeutic agent can be memantine, analgesics including acetametophin and codeine, morphine, ibuprofen, and naproxen, or antidysrhythmics including digoxin, furosemide, hydrochlorothiazide, and metolazone.

For schizophrenia or schizotypal disorders, an additional therapeutic agent can be antipsychotics including aripiprazole, asenapine, brexpiprazole, buspirone, cariprazine, chlorpromazine hydrochloride, clozapine, haloperidol, iloperidone, loxapine, lumateperone, lurasidone hydrochloride, molindone hydrochloride, olanzapine, paliperidone, perphenazine, prochlorperazine, quetiapine, risperidone, thiothixene, trifluoperazine, or ziprasidone.

For hypogonadotropic hypogonadal syndromes, an additional therapeutic agent can be androgens including testosterone,methyltestosterone, and fluoxymesterone, gonadotrophins including chorionic gonadotropin and follitropin, or Teriparatide.

For disorders associated with neurotoxicants, an additional therapeutic agent can be stimulants including methamphetamine, methylphenidate, dexmethylphenidate, amphetamine, dextroamphetamine, lisdexamfetamine, isdexamfetamine dimesylate, bupropion, venlafaxine, and imipramine, risperidone, lithium and methylphenidate, carbamazepine, quetiapine, or anticonvulsants such as citalopram, escitalopram, fluoxetine, paroxetine, or sertraline.

For attention deficit hyperactivity disorder, an additional therapeutic agent can be stimulants including methamphetamine, methylphenidate, dexmethylphenidate, amphetamine, dextroamphetamine, lisdexamfetamine, isdexamfetamine dimesylate, bupropion, venlafaxine, and imipramine, central alpha-2 adrenergic agonists including clonidine, guanfacine, or norepinephrine reuptake inhibitors including atomoxetine.

A group II metabotropic glutamate receptor modulator can be administered before or after the administration of the additional therapeutic agent. In some embodiments, the group II metabotropic glutamate receptor modulator and the additional therapeutic agent are administered concurrently, or in close temporal proximity (e.g., there may be a short time interval between the administrations, such as during the same treatment session). In some embodiments, there may be greater time intervals between the administrations, such as during the same or different treatment sessions.

In some embodiments, methods associated with the disclosure comprise administering an effective amount of a group II metabotropic glutamate receptor modulator alone or in combination with an additional therapeutic agent to a subject who has been identified as having a loss-of-function mutation in the PTCHD1 gene. In some embodiments, methods comprise determining whether a subject has a loss-of-function mutation in the PTCHD1 gene, and administering an effective amount of a group II metabotropic glutamate receptor modulator alone or in combination with an additional therapeutic agent to the subject if the subject has a loss-of-function mutation in the PTCHD1 gene.

### General Techniques

The practice of the present disclosure will employ, unless otherwise indicated, conventional techniques of molecular biology (including recombinant techniques), microbiology, cell biology, biochemistry and immunology, which are within the ordinary skill in the art (e.g., as disclosed in: Molecular Cloning: A Laboratory Manual, fourth edition (Green, et al., 2012 Cold Spring Harbor Press); Oligonucleotide Synthesis (M. J. Gait, ed., 1984); Methods in Molecular Biology, Humana Press; Cell Biology: A Laboratory Notebook, Vol. 3 (J. E. Cellis, ed., 2005) Academic Press; Animal Cell Culture (R. I. Freshney, ed., 1987); Introduction to Cell and Tissue Culture (Q. P. Mather and P. E. Roberts, 1998) Plenum Press; Cell and Tissue Culture: Laboratory Procedures (A. Doyle, J. B. Griffiths, and D. G. Newell, eds., 1993-8) J. Wiley and Sons; Methods in Enzymology (Academic Press, Inc.); Handbook of Experimental Immunology (D. M. Weir and C. C. Blackwell, eds.); Gene Transfer Vectors for Mammalian Cells (J. M. Miller and M. P. Calos, eds., 1987); Short Protocols in Molecular Biology (F. M. Ausubel, et al., eds., 2002); PCR: The Polymerase Chain Reaction, (Mullis, et al., eds., 1994); Current Protocols in Immunology (J. E. Coligan et al., eds., 1991); Short Protocols in Molecular Biology (Wiley and Sons, 1999); Immunobiology (C. A. Janeway and P. Travers, 1997); Antibodies (P. Finch, 1997); Antibodies: a practical approach (D. Catty, ed., IRL Press, 1988-1989); Monoclonal antibodies: a practical approach (P. Shepherd and C. Dean, eds., Oxford University Press, 2000); Using antibodies: a laboratory manual (E. Harlow and D. Lane (Cold Spring Harbor Laboratory Press, 1999); The Antibodies (M. Zanetti and J. D. Capra, eds., Harwood Academic Publishers, 1995). It is believed that one skilled in the art can, based on the above description, utilize the present invention to its fullest extent. The following specific embodiments are, therefore, to be construed as merely illustrative, and not limitative of the remainder of the disclosure in any way whatsoever. All publications cited herein are incorporated by reference for the purposes or subject matter referenced herein.

### EXAMPLES

In order that the invention described herein may be more fully understood, the following examples are set forth. The examples described in this application are offered to illustrate the systems and methods provided herein and are not to be construed in any way as limiting their scope.

### Example 1: Study Designs and Analyses on Group II mGluRs' Therapeutic Mechanisms

LY379268, LY404039, LY395756, RO 645229 and ML337 were purchased from Tocris Bioscience. Mavalon 63 and AZD8529 were synthetized in-house. Picrotoxin was purchased from Sigma-Aldrich. All stock solutions were prepared with Milli-Q water (Millipore) or DMSO and stock aliquots were stored at −20° C. until use.

In order to examine various group II mGluRs' therapeutic mechanisms, transgenic animal models were used for conducting experiments. In general, all the behavior experiments were done in healthy age-matched male mice between 2-5 months with C57Bl/6J background, Ptchd1Y/− (knockout) and Ptchd1Y/+ (wild type). In all the compared groups, a counterbalancing strategy was employed to ensure that both genotypes were being tested at equal frequencies throughout the testing day. For drug studies, mice were chosen randomly to be included in the vehicle-treated versus drug-treated groups. Experimenters were blind to genotype until the data analysis was conducted and finished. Breeding colonies were maintained and two Ptchd1+/−female mice and one male mouse with C57Bl/6J background were housed in each cage. Mice were housed with a standard 12 h light/12 h-dark cycle (lights on at 07:00, lights off at 19:00) with ad libitum food and water. All animal studies were conducted in accordance with NIH guidelines and approved by the MIT Institutional Animal Care and Use Committee.

For EEG recording studies, 8- to 12-week-old male mice with C57Bl/6J background, Ptchd1Y/− (knockout) and Ptchd1Y/+ (wild type) were deeply anesthetized with a 1% to 1.5% isoflurane. A screw electroencephalography (EEG) electrode (catalog no. 8403; Pinnacle Technology, Inc.) was implanted into the skull above the primary somatosensory cortex, S1 (AP: −1.8 and ML: +3.0 mm) using a stereotaxic device (David Kopf Instruments). A reference electrode was screwed in the occipital region of the skull and stainless steel electromyography (EMG) electrodes were placed in the nuchal muscle. All the electrodes were connected to a head-mount (catalog no. 8201-SS; Pinnacle Technology) and secured by using dental cement. The mice were allowed to recover for at least 2 weeks. Spontaneous EEG/EMG signals were recorded for 12 hours with a differential amplifier (8200-K1-SL amplifier; Pinnacle Technology). All signals were digitized at a sampling frequency of 1,000 Hz, filtered (1-100 Hz bandpass for EEG; 10-1 kHz bandpass for EMG), and acquired using Sirenia Acquisition program (Pinnacle Technology). Data were analyzed offline using Matlab (R2016a; MathWorks). The spectral power was calculated in 0.5-Hz bins (fast Fourier transform with Hamming window) with artifact-free 4-hour EEG signals from each animal. The power for the spectral band was normalized in reference to the power of the entire EEG spectrum (1-100 Hz) in each animal and averaged across all the animals in the same treatment or drug condition.

The TRN region was dissected for conducting the single cell RNAseq analysis. In brief, a transgenic Pvalb-tdTomato mouse line expressing tdTomato in TRN neurons was used. Fluorescence-aided micro-dissection of tdTomato-positive TRN tissues under a stereo fluorescence microscope was performed. To minimize the batch effect due to gender difference, only female mice were included in this study to reduce gene expression bias. Female mice at 13-15 weeks p.n. were deeply anesthetized with isoflurane followed by cervical dislocation and decapitation for tissue harvesting. Brains were rapidly removed and placed in ice-cold cutting solution containing 194 mM sucrose, 30 mM NaCl, 4.5 mM KCl, 1.2 mM NaH₂PO₄, 0.2 mM CaCl₂, 2 mM MgCl₂, 26 mM NaHCO₃, and 10 mM D-(+)-glucose saturated with 95% O₂ and 5% CO₂, pH 7.4, 320-340 mOsm/L. A sagittal slice (thickness of 250 μm) series covering the whole TRN section was prepared using a slicer (VT1200 S, Leica Microsystems, USA) and placed under a fluorescent stereomicroscope for microdissection. Dissected TRN regions were placed into ice-cold RNAlater (Sigma) to preserve samples. The samples were transferred to 4° C. for storage overnight followed by storage at −80° C. until further processing.

For performing the open field test, mice were allowed to habituate to the behavioral testing room for 1 hour before the experiment. The locomotor activity in mice was evaluated over a 90 minute period in an automated Omnitech Digiscan apparatus (AccuScan Instruments). Locomotor activity was assessed as total distance traveled (m). For drug treatment experiments, mice received an intraperitoneal injection 15 minutes before the assay and then where placed kindly in the left corner, head facing into the corner.

Behavioral Training and Testing was conducted. In brief, behavioral training and testing took place in grid-floor mounted, custom-built enclosures made of acrylic plastic (dimensions in cm: length: 15.2; width: 12.7; height: 24). All enclosures contained custom-designed operant ports, each of which was equipped with an IR LED/IR phototransistor pair (Digikey, Thief River Falls, Minn.) for nose-poke detection. Trial initiation was achieved through an initiation port mounted on the grid floor 6 cm away from the ‘response ports’ located at the front of the chamber. A pair of electrostatic speakers (Tucker Davis Technologies) producing the auditory stimuli were placed outside of the training apparatus and sound stimuli were conveyed via cylindrical tubes to apertures located at either side of the initiation port, allowing delivery of stereotypical stimuli across trials. All stimuli across tasks were generated by a TDT Rx8 sound system (Tucker-Davis Technologies, Alachua, Fla.). Sound stimuli were recorded and assessed for intensity using a prepolarized icp array microphone (PCB Piezotronics, Depew, N.Y.) after which frequency production was equalized using a software-based calibration via SigCalRP (Tucker-Davis Technologies, Alachua Fla.).

Response ports were separated by 1 cm divider walls and each was capable of delivering a milk reward (10 μl evaporated milk delivered via a single-syringe pump (New Era Pump Systems, Farmingdale, N.Y.) when a correct response was performed. For the auditory GO/NO GO task environment, response and reward ports were dissociated as explained in the relevant section below. Access to all response ports was restricted by vertical sliding gates that were moved via a servo motor (Tower Hobbies, Champaign, Ill.). The TDT Rx8 sound production system (Tucker Davis Technologies, Alachua, Fla.) was triggered through MATLAB (MathWorks, Natick, Mass.), interfacing with a custom written software running on an Arduino Mega (Ivrea, Italy) for trial logic control. 50 ms continuous interruption of the IR beam in the initiation port was required before a trial initiation was started, after which the animal was still required to hold through the delay period (typically 400 ms) as well as the sound stimulus presentation. This ensured that the animal's head was properly positioned to hear the stimuli. Mice were food restricted to 85-90% of their ad libitum body weight before training.

For conducting auditory GO/NO GO test, a total of 4 control, 5 Ptchd1 KO mice were trained on this task. No differences were observed in learning between these groups. In training and testing, mice initiated each trial by holding their snout in an initiation port for a 400 ms delay period. After successful initiation, a pure tone stimulus was played for 100 ms from speakers on both sides of the initiation port at an intensity of 60 dB. A 20 kHz tone signaled a target, “GO” response, while frequencies 16 and 24 kHz signaled a non-target “NO GO” response. The pure tone stimuli were pseudo-randomly varied on a trial by trial basis, with trials divided between the “GO” stimulus (˜40% of trials) and two “NO GO” stimuli (16 and 24 kHz, ˜30% of trials per frequency).

The sequence of “NO GO” stimuli followed a pre-determined pseudorandomized sequence which was shifted across testing sessions. After the stimulus presentation, the response port was made accessible for a 2.5 second trial period. In “GO” trials, the mouse was required to poke in this response port within the trial period (a “Hit”) in which case a reward port directly underneath the response port became accessible, and the reward was delivered. For a “Miss” in which the mouse failed to poke within the time limit, the reward port was not made accessible. For a “correct rejection,” withholding for the full 2.5 seconds when the “NO GO” stimulus was played, the reward port was made accessible. For a “False Alarm” response on a “NO GO” trial, the reward port was not made accessible and the next trial was delayed by a 15 second time-out period. For noise masking experiments, white noise was added to pure tone stimuli at fixed SNR ratios as labeled in each relevant figure. In each session in which noise was presented without cueing, subsets of 20-40% of trials were randomly masked by noise (average session length=212 trials).

For behavioral analysis, experiments examining sound detection were assessed using the percent correct performance. Sessions were only included if performance on max intensity was greater than 60% correct.

For conducting Florescent In Situ Hybridization (FISH) analysis, 6-8 week old mice were deeply anesthetized with isoflurane, decapitated, the brains were rapidly dissected out and frozen in blocks using Optimal Cutting Temperature (O.C.T. Tissue-Tek) medium in an isopropyl ethanol/dry ice bath. Serial sections of the samples were cut at 16 μm thickness using a cryostat (Leica CM 1850), adhered to SuperFrost Plus microscope slides (Fisher Scientific, 12-550-15), and stored at −80° C. until use. Samples were immediately fixed in 4% paraformaldehyde for 30 minutes at 4° C. and stained on the slide according to the Advanced Cell Diagnostics RNAscope Fluorescent Multiplex Assay (ACD, 320850) protocol. Samples were then stained for Pvalb (ACD, 421931), Grm3 with antisense probes, and mounted with a coverslip using Vectashield hardset antifade mounting medium with DAPI (Vector Laboratories, H-1500). Z-stack serial images were taken using a Nikon Ti Eclipse inverted microscope equipped with an Andor CSU-W1 confocal spinning disc unit and an Andor DU-888 EMCCD together with 20×/0.75 NA air objective lens. Microscope and camera settings were consistent for all imaging procedures.

For conducting slice electrophysiology analysis, whole-cell patch-clamp recordings were performed using horizontal slices for the rebound bursting assays and angled-coronal thalamocortical slices for the synaptic optogenetics experiments (Agmon et al., 1991). 21-35 day old male C57BL6 mice and Ptchd1 Y/− (knockout) mice were used. Animals were transcardially perfused under isoflurane anesthesia with an ice-cold cutting solution (194 mM Sucrose, 30 mM NaCl, 4.5 mM KCl, 1.2 mM NaH₂PO₄, 10 mM Glucose, 2 mM MgCl₂, 0.2 mM CaCl₂, and 26 mM NaHCO₃). Mice were then decapitated and the brains were carefully dissected out. Both cerebellum and olfactory bulb were removed from the brain and were placed and glued onto the stage of a Leica Vibratome with the ventral surface down and the pial surface towards the blade. 250 micron thick slices containing the TRN were then collected and recovered in regular artificial cerebrospinal fluid (aCSF; 119 mM NaCl, 2.3 mM KCl, 1 mM NaH₂PO₄; 11 mM Glucose, 1.3 mM MgCl₂, 2.5 mM CaCl₂), 26 mM NaHCO₃) heated to 32° C. for 10 minutes and then at room temperature for at least 1 hr. All buffers were continuously bubbled with 95% O₂/5% C02. Slices were then transferred to a submersion recording chamber where they were perfused with aCSF at a rate of 2 ml/min at room temperature. Patch recording pipettes were pulled using a Flaming/Brown micropipette puller (Sutter Instruments) and Borosilicate glass (KG33, King Precision Glass). The DC resistances were ˜4-6 MΩ. Potassium methanesulfonate based internal solution (140 mM KMeSO₃; 10 mM KCl; 10 mM HEPES; 0.1 mM EGTA; 4 mM Mg-ATP; 0.2 mM Na-GTP; 10 mM hosphocreatine) was used. Neurons were visualized using infrared differential interference contrast (IR-DIC) microscopy (Olympus, BX51W1) with an upright microscope digital camera.

The recordings were made with a microelectrode amplifier with bridge and voltage clamp modes of operation (Multiclamp 700B, Molecular Devices, San Jose, Calif., USA). Signals were low-pass filtered at 2 kHz and sampled at 10-20 kHz with a Digidata 1440A (Molecular Devices, San Jose, Calif., USA). Data were stored on a computer for subsequent off-line analysis. Cells with the series resistance (Rs, typically 8-12 MΩ) changed by >20% were excluded for data analysis. In addition, cells with Rs more than 20 MG at any time during the recordings were discarded.

Following electrophysiological recordings, the slices were fixed overnight in 4% paraformaldehyde at 4° C. After 3 washes in PBS for 10 min, brain slices were permeabilized with 0.25% Triton X-100 in PBS for 15 min, washed 3 times for 5 min in PBS and blocked 1 h in 3% BSA, 0.25% Triton X-100 in PBS (blocking buffer) for 1 h at room temperature. Biocytin-filled cells were visualized by incubating sections in 1:500, 488 streptavidin (Invitrogen) in PBS blocking buffer for 4 hrs. After washing, all sections were mounted in Vectashield (Vector Laboratories).

To study the changes in the acute somatosensory thalamocortical or horizontal brain areas, 21-25 days old Nex-Cre or VgluT2-Cre mice were anesthetized with isoflurane while placed in a small animal stereotaxic apparatus (David Kopf Instruments, CA, USA) and were injected bilaterally with 280 nl of mcherry-dflox.hChR2.EF1.AAV1 using a Nanoject (Drummond Scientific, Broomall, Pa.) via glass pipettes with 20-30 sm diameter tips in somatosensory cortex (S1) and ventrobasal thalamic complex (VB). Coordinates from the bregma for S1: AP −0.7, ML 2.4 and DV 1.2 and for VB: AP −1.6, ML 1.5 and DV 3.5. The pipette was held in the final position for 10 min after injection before withdrawal from the brain. The incision was closed with vetbond and after surgery mice were injected with analgesics (SR-Bupronex 1 mg/kg) every 72 hours or as needed. Mice were recovered on a heat pad in a clean cage and were allowed at least 7 days of recovery before electrophysiological recordings were performed. All experimental mice were group-housed pre and post-surgery.

After allowing 1-2 weeks for mcherry-dflox.hChR2.EF1.AAV1 expression, acute somatosensory thalamocortical or horizontal brain slices were freshly obtained for in vitro recording and optogenetic stimulation (250 um thick, thalamocortical slices containing TRN from angled-coronal sections; Agmon and Connors, 1991) for optogenetic analysis. In brief, patch clamp recordings in voltage clamp configuration were performed while the Cortico-TRN or Thalamic-TRN pathway was activated by applying a blue LED (400-450 nm) light pulses (Lumen 300-LED Fluorescence Excitation Illumination System). For synaptic stimulation, neurons were held at −70 mV while 0.1-1.5 ms flashes were delivered directly to the recorded cell soma delivered through the 40× water immersion objective. Tissues from thalamus and globus pallidus, which are adjacent to TRN, as controls, were collected.

For performing confocal imaging analysis, the fluorescent images were taken as z-stacks on a Nikon Eclipse Ti-E inverted confocal equipped with Andor CSU-W1 spinning disc unit and EMCCD camera (Andor, iXon Ultra 888). Excitation laser lines were 405 nm (DAPI), 488 nm (Alex Fluor 488) and 561 nm (Alexa Fluor 568, mCherry). Z-stacks were max projected to get a single section confocal image and exported to TIFF file format.

### Example 2: mGluR₃ is Highly Expressed in the TRN and the Activation of mGluR₃ Facilitates the Repetitive Burst Firing of TRN Neurons

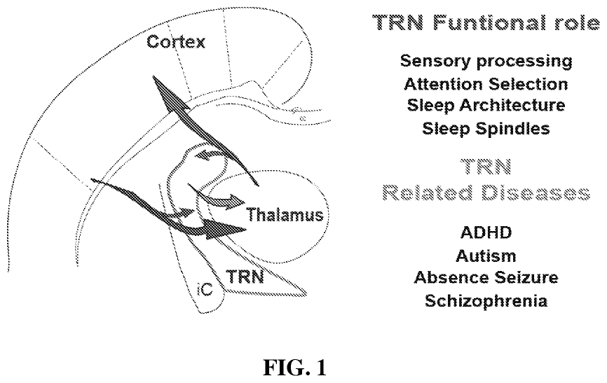

The TRN is a central control unit in the thalamocortical circuit, receiving input from both the thalamus and cortex while only inhibiting thalamic targets (FIG. 1). It is anatomically segregated into subnetworks that project to spatially discrete thalamic targets, allowing the TRN to influence multiple levels of functional thalamic organization. These include global arousal and specific cognitive states such as attention. A well-established characteristic of TRN neurons is their intrinsic oscillatory firing property that plays a role in regulating TC circuit rhythms, sleep architecture and attention. Depending on their resting membrane potential, these neurons can fire in two distinct modes upon receiving synaptic input (Pinault et al., 2004). At depolarized membrane potential, the TRN neurons exhibit tonic firing. When hyperpolarized, the TRN neurons generate repetitive rebound bursting mainly depending on interactions of “low-threshold” T-type Ca2+ transients and small conductance calcium-activated K+(SK) channels. Acute brain slice recordings identified a reduced bursting phenotype of TRN neurons in Ptchd1 KO model (Wells et al., 2016). It was also reported that pharmacological rescue of this biophysical dysfunction selectively alleviated the ADHD-related behaviors.

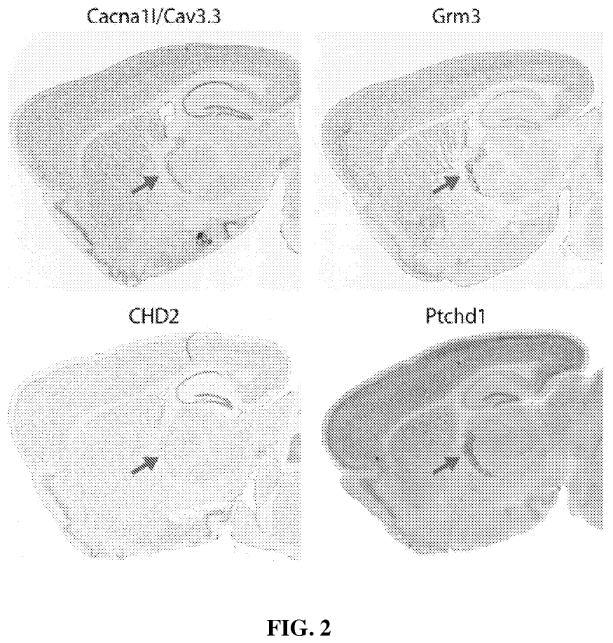

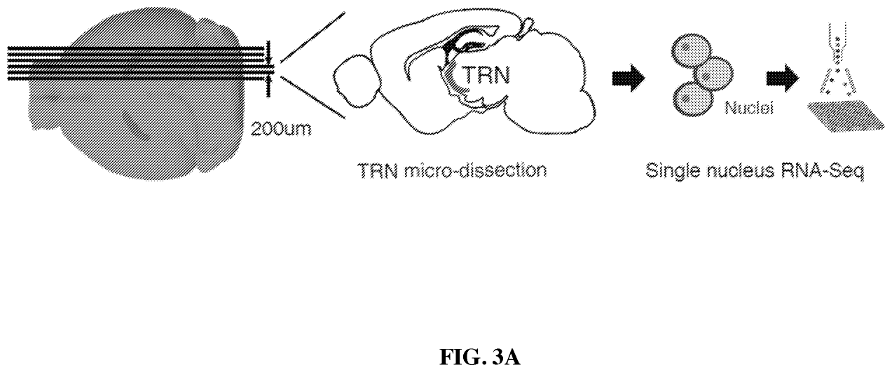

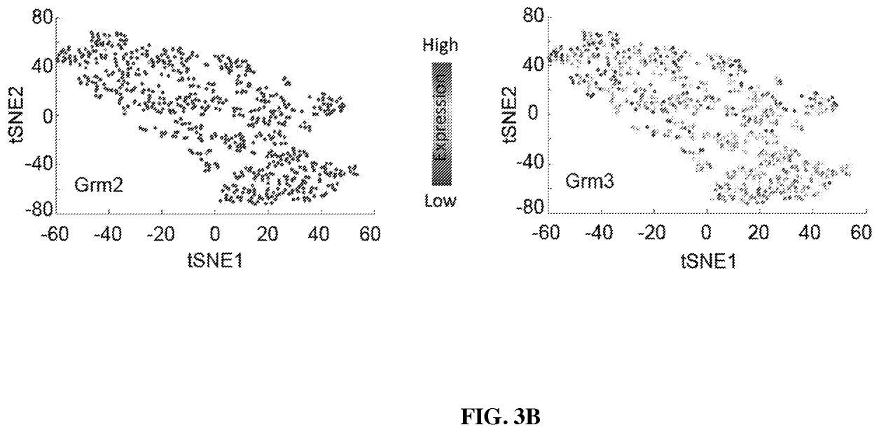

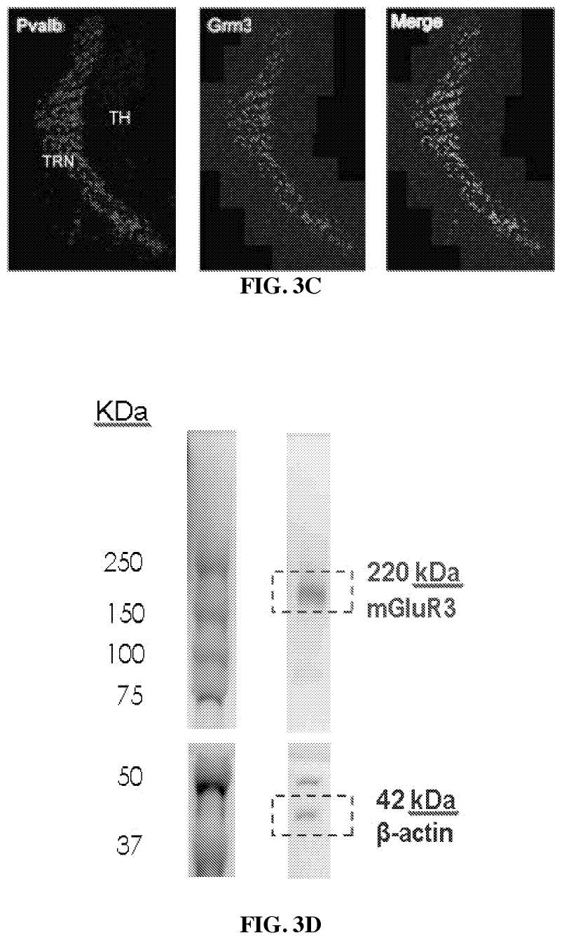

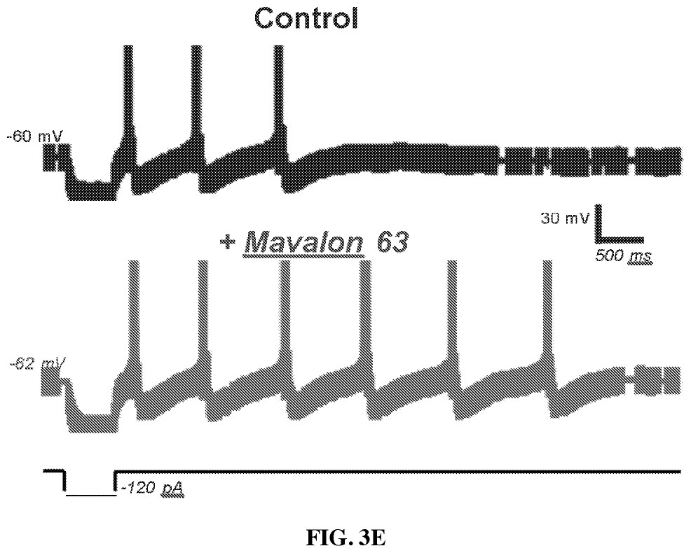

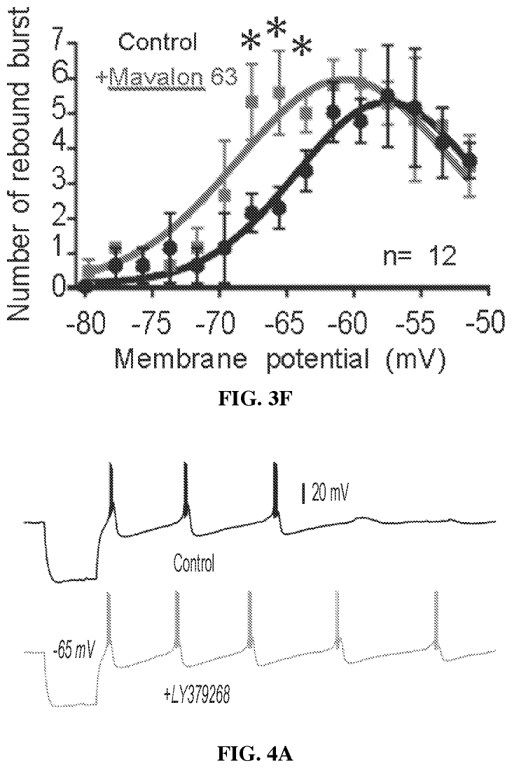

Brain tissue staining showed that several neurodevelopmental disorder-linked genes are highly expressed in the TRN, including mGluR₃ (labeled as Grm3), PTCHD1, Cacna11, and CHD2 (FIG. 2). Enriched expression of mGluR₃ in the TRN was confirmed by single nucleus RNA-seq of the TRN (FIG. 3A and FIG. 3B). T-distributed stochastic neighbor embedding (t-SNE) of single nuclei showed that mGluR₃ was expressed in the TRN, which was confirmed by RNA-fluorescence in situ hybridization (FISH) analysis (FIG. 3C) and Western blot analysis (FIG. 3D). Taken together, these results demonstrated that mGluR₃ is highly expressed in the TRN.

Regulation of rebound bursting in TRN neurons by selective activation of mGluR₃ was examined. Surprisingly, the newly developed mGluR₃ PAM, Mavalon 63, as shown in FIGS. 3E and 3F, produced a significant increase in repetitive rebound bursting when compared with WT and Ptchd1 KO mice in the absence of the treatment of Mavalon 63. These results show that mGluR₃ activation can enhance intrinsic bursting properties of TRN neurons, and may reverse TRN dysfunction.

### Example 3: Group II Metabotropic Glutamate Receptor Modulators Alter Rebound Burst Firing in TRN Neurons

TRN neurons are known to display dual firing modes depending on their resting membrane potential: tonic and burst firing. While tonic firing refers to regular sodium spike trains at depolarized membrane potential, burst firing is characterized by repetitive “low-threshold” T type Ca2+ transients (mediated by CaV3.3 channel) crowned by high-frequency sodium spikes when hyperpolarized.

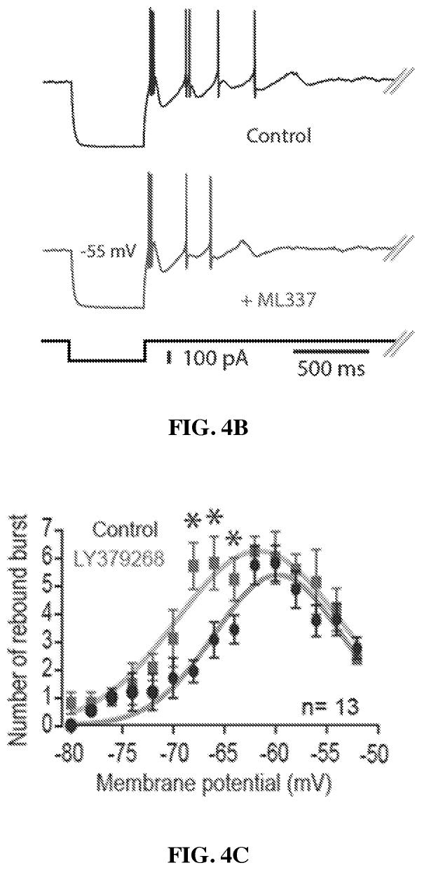

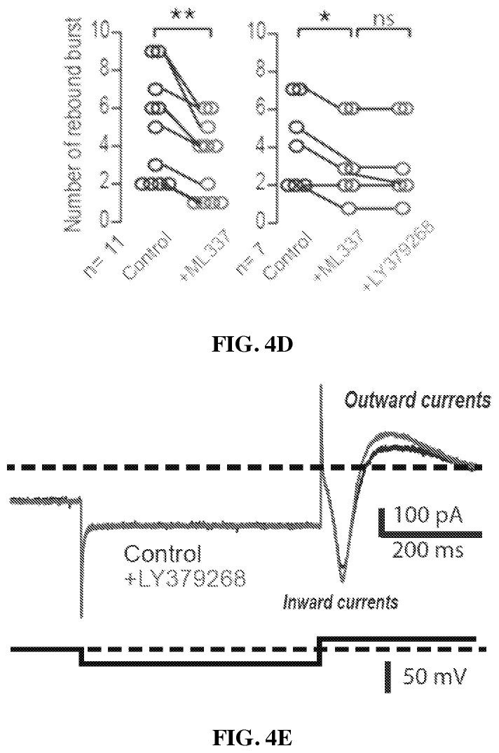

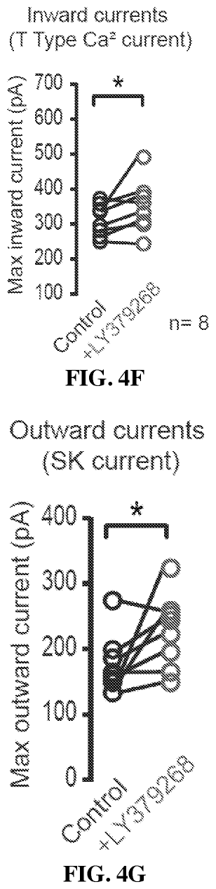

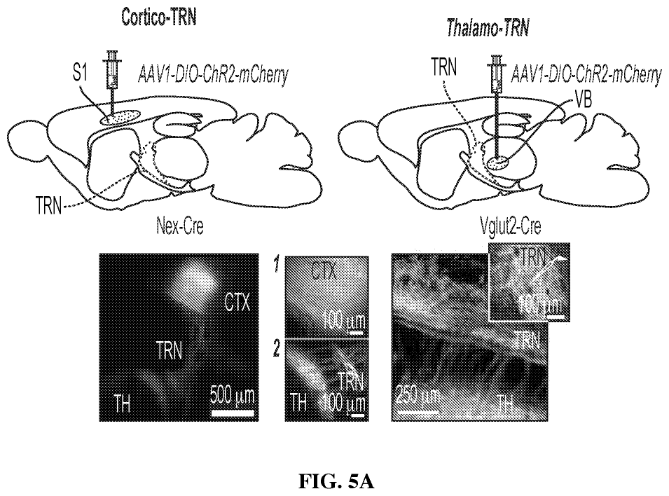

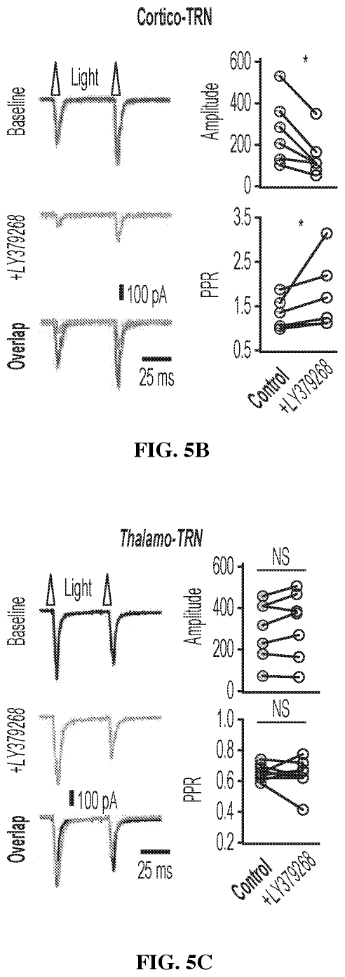

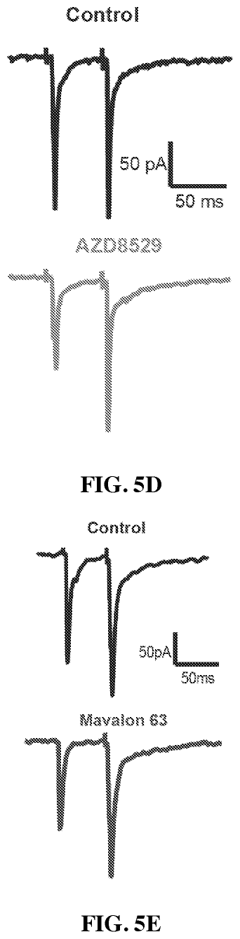

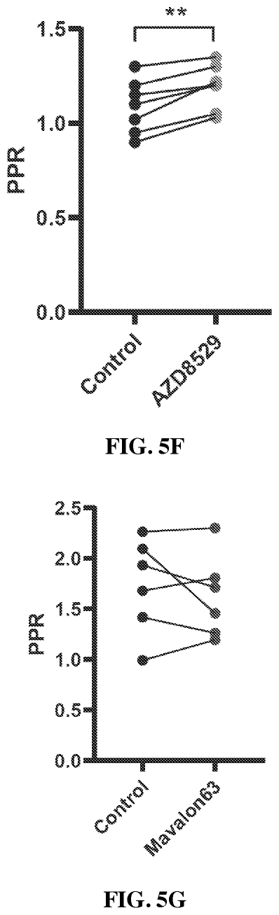

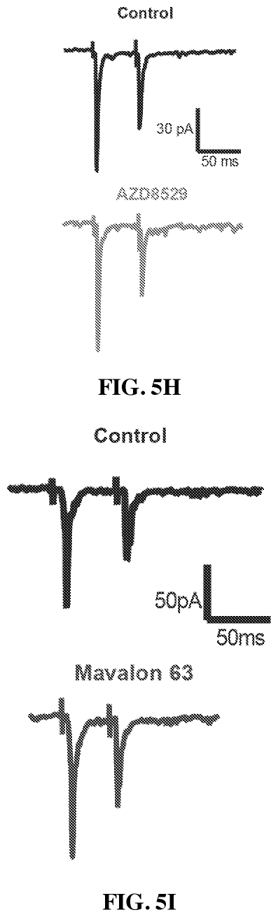

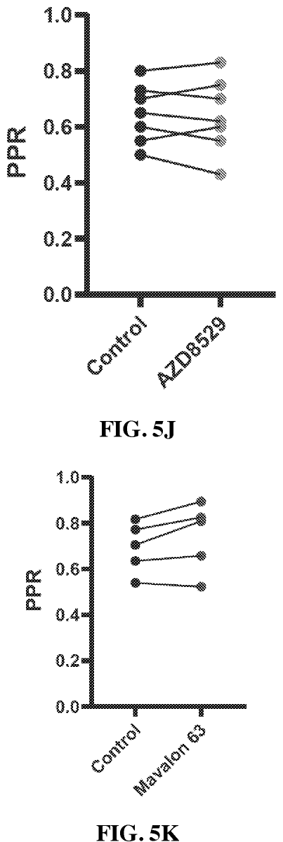

Whether this characteristic TRN burst firing activity is affected by activation of the group II metabotropic glutamate receptors, including mGlu₂ and mGlu₃, was examined using the group II metabotropic glutamate receptor agonist, LY379268. A significant increase in the repetitive rebound bursting after the addition of LY379268 compared to baseline levels in acute brain slices acquired from wild-type (WT) mice was observed (FIGS. 4A and 4C-4D). By contrast, a decrease in the repetitive rebound bursting was observed after the addition ML337, which is a negative allosteric modulator (NAM) (FIGS. 4B and 4D). As shown in FIG. 4E-4G, the treatment of LY379268 significantly increased the max inward currents and the max outward currents compared with the control group without LY379268. Dissection of mGluR_{2/3} activity on the thalamocortical synaptic actions by optogenetic activation of either layer 6 pyramidal neuron or VB thalamic relay neurons demonstrated a selective regulation of cortico-reticular synapses but not thalamic-reticular synapses (FIGS. 5A-5C).

Taken together, these results demonstrate that group II metabotropic glutamate receptor modulators regulate TRN burst firing activity and cortico-reticular synapses.

### Example 4: Activation of mGluR_{2/3} Rescued Rebound Bursting in a Mouse Model of Neurodevelopmental Disorders

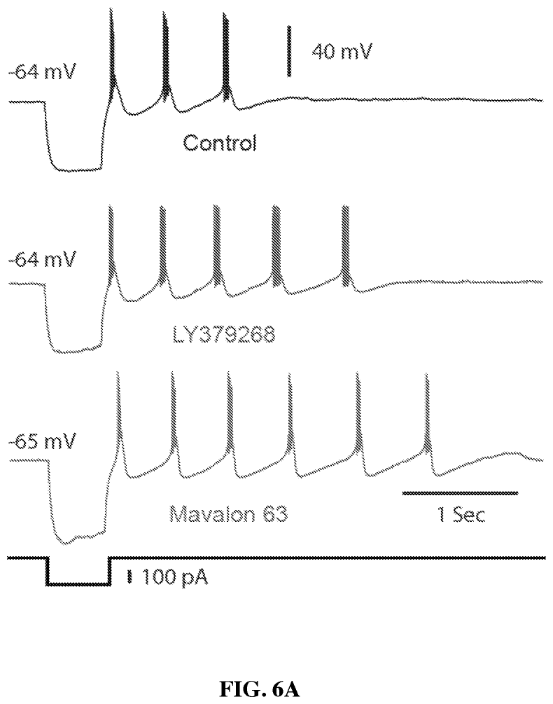

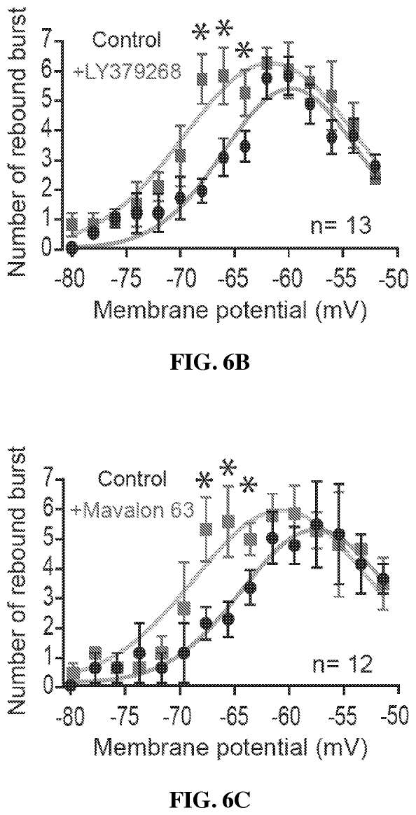

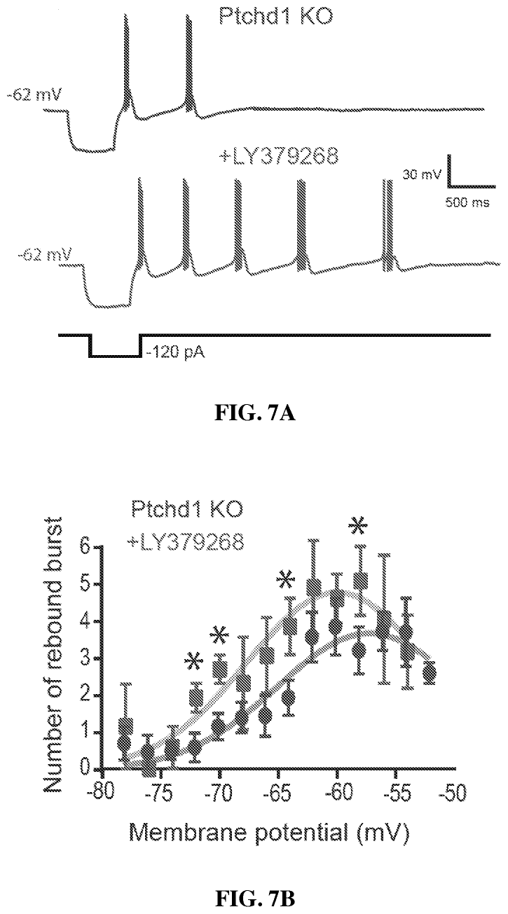

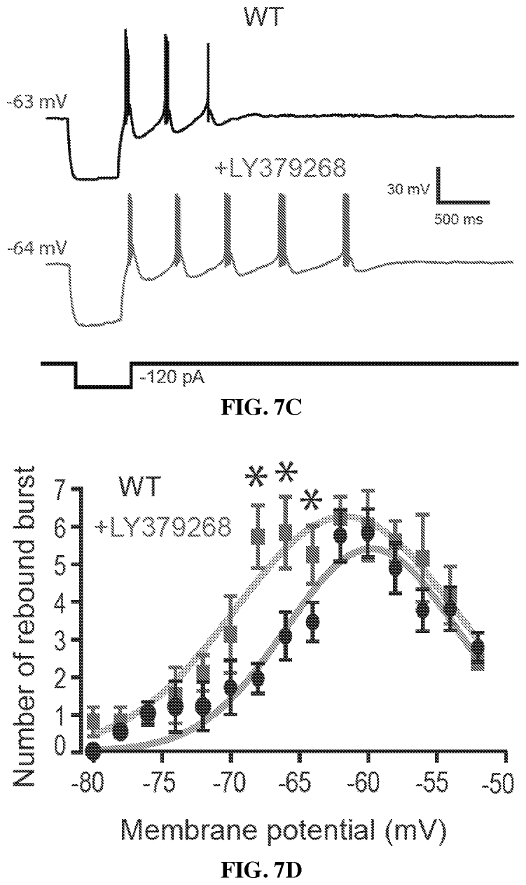

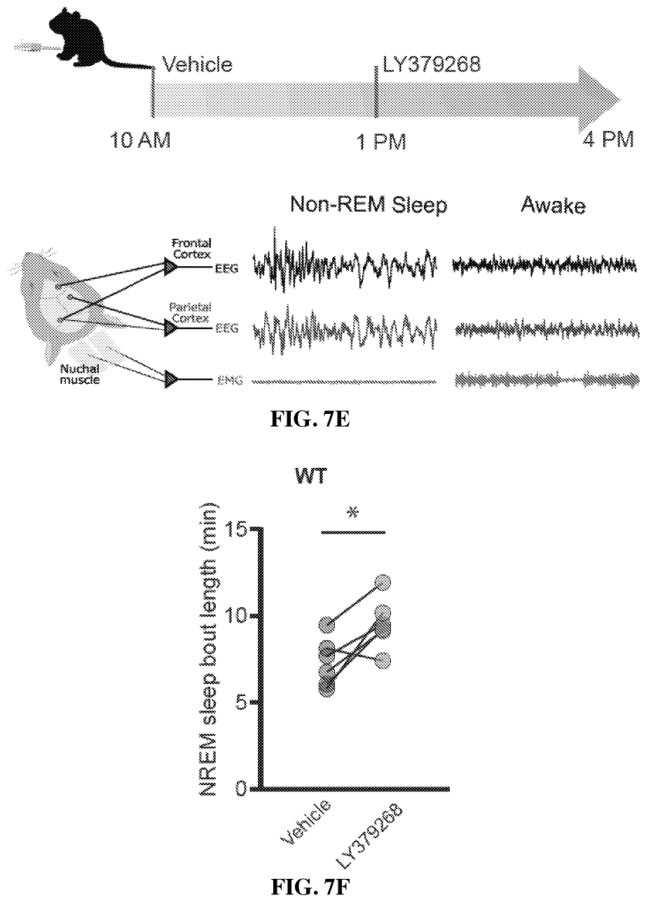

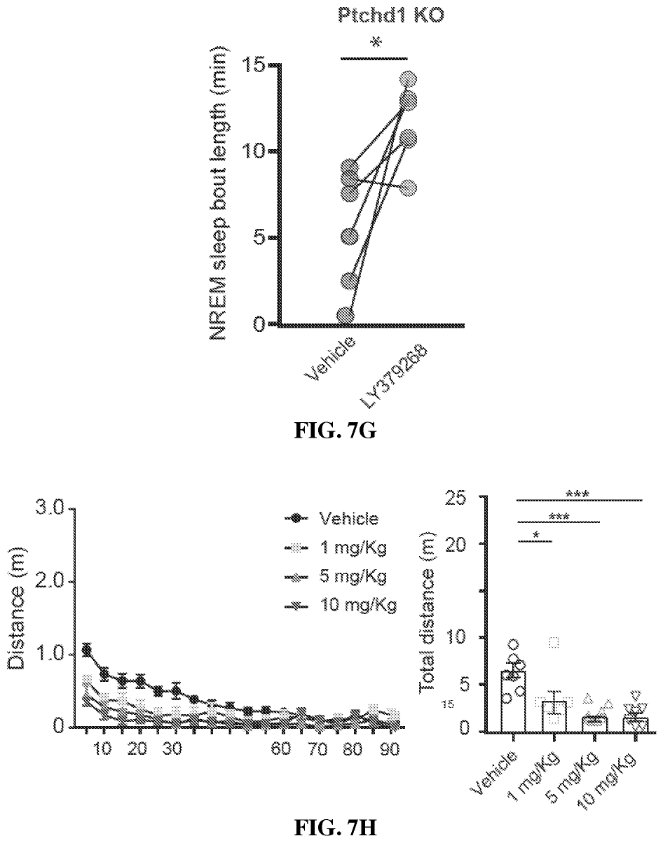

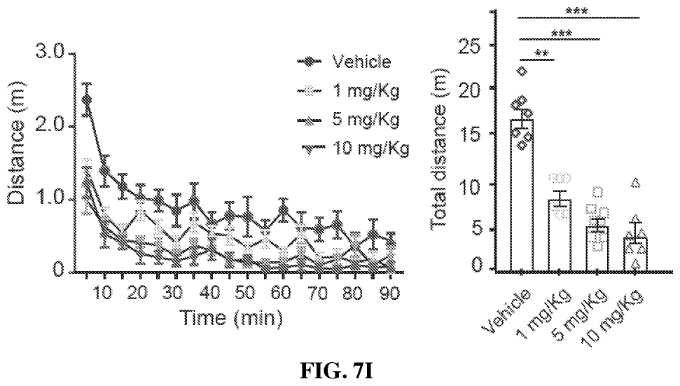

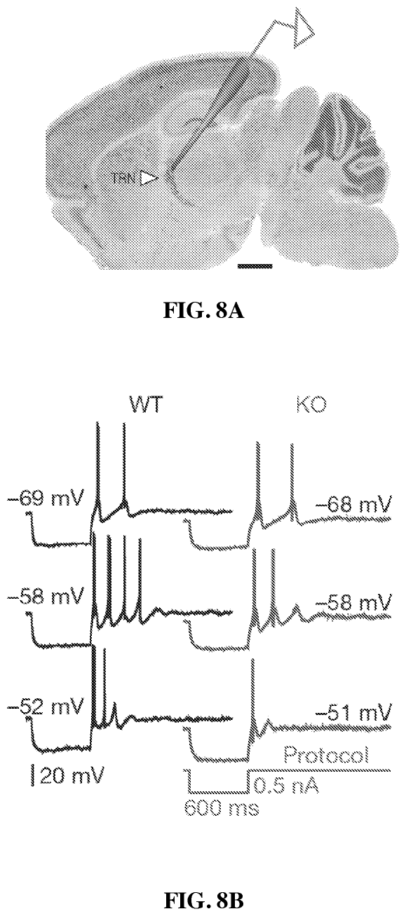

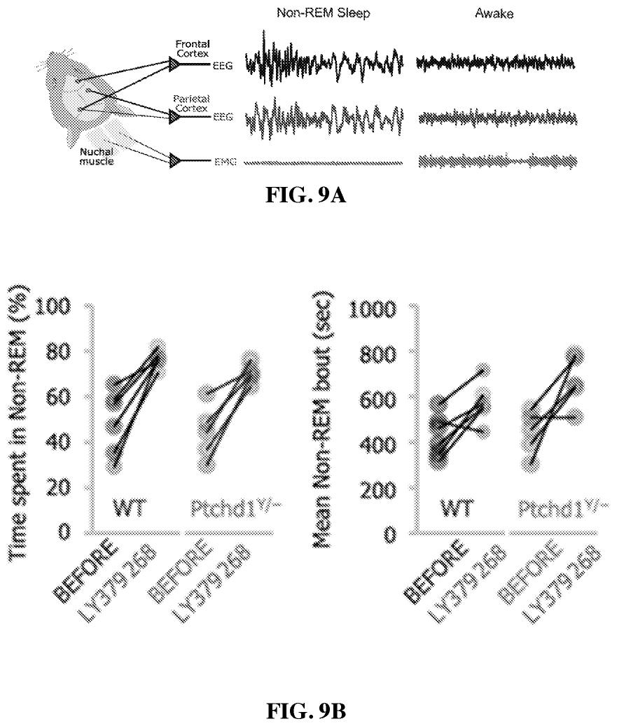

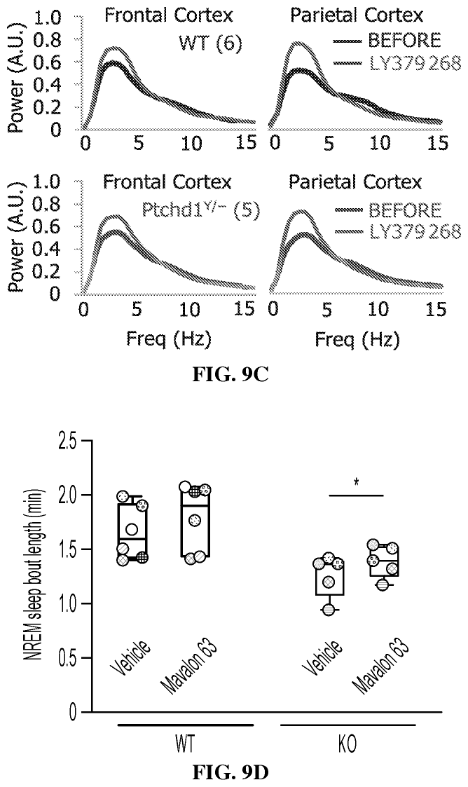

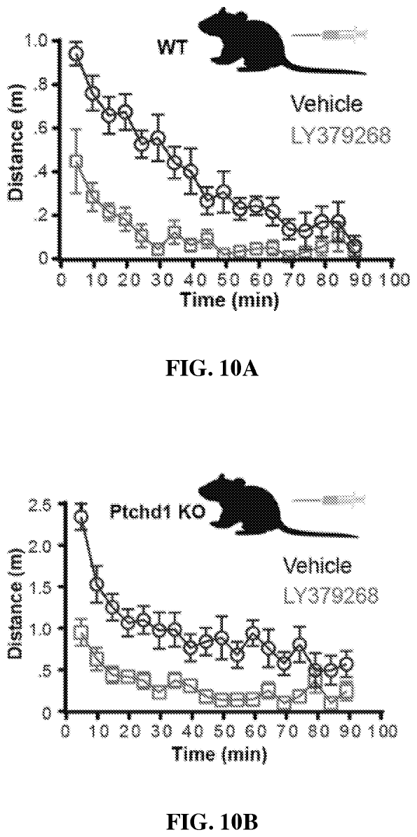

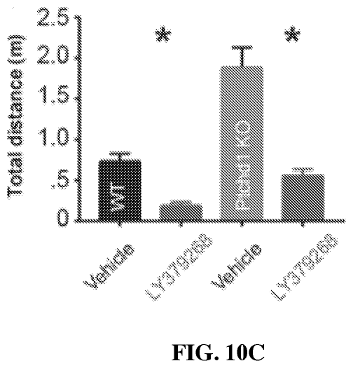

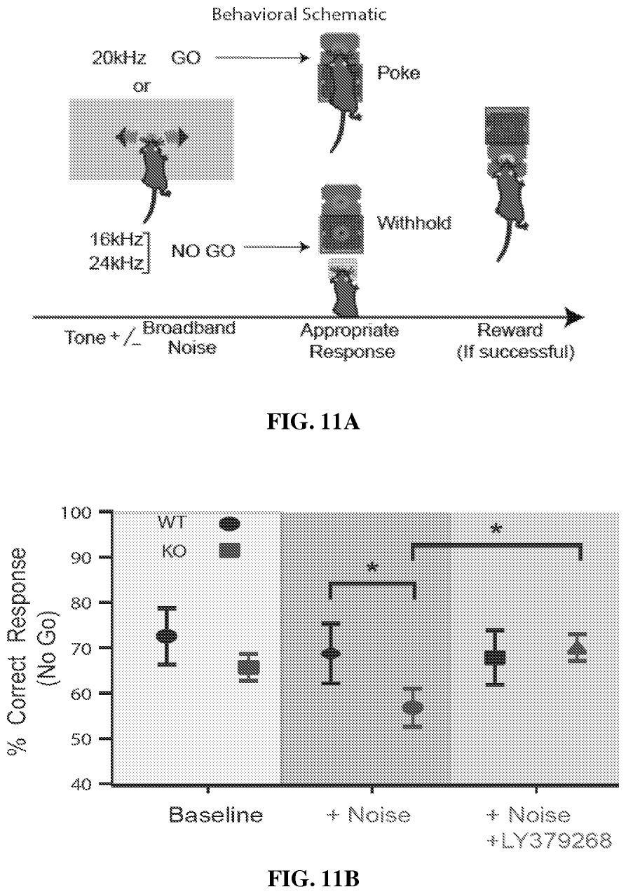

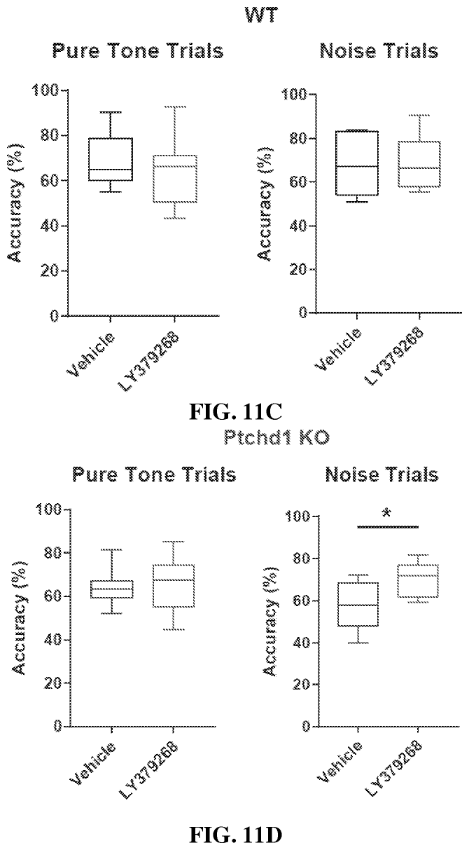

Augmentation of mGluR_{2/3} activity was sufficient to correct the reduced rebound bursting in a mouse model of neurodevelopmental disorders (e.g., Ptchd1 mutant mouse model) (FIGS. 6A-6C and 7A-7B). A Ptchd1 mutant mouse displayed reduced rebound bursting compared to WT mice (FIGS. 9A-9B). Pharmacological modulation of mGlu₂/3 activity rescued sleep deficits (FIGS. 9A-9C), hyperactivity (FIGS. 10A-10C), and attention deficits (FIGS. 11A-11B) in Ptchd1 knockout mice. Attention deficits are discussed further below in Example 8.

Taken together, these results demonstrated rescue of behavioral deficits by boosting mGlu_{2/3} activity in a mouse model of developmental disorders.

### Example 5: Effect of mGluR2 and mGluR3 on Synaptic Actions in TRN Medicated TC Circuit

The TRN receives collateral inputs from both ascending thalamo-cortical and descending cortico-thalamic neuron axons originating mainly from Layer 6 cortical neurons. TRN neurons send the inhibitory outputs to thalamic relay cells, providing the major source of inhibition to thalamus. Studies were conducted to evaluate the differential regulation of cortico-TRN synapses versus thalamo-TRN synapses by group II mGluRs activity. Studies were also conducted to evaluate whether mGluR2 and mGluR3 exhibit distinct effect on synaptic transmission in TRN mediated TC circuit.

In order to test the potential differential regulation by mGluR2 and mGluR3, a Nex-Cre and Vglut2-Cre mouse line was used to allow for genetic access to layer 6 cortical neurons or thalamic relay neurons. Optogenetic circuit-mapping by expressing the light-activated Channelrhodopsin via injections of mcherry-dflox.hChR2.AAV1 (FIG. 5) into the somatosensory primary cortex (S1) or the ventrobasal thalamus (VB) was performed. The whole cell patch-clamp recordings of optically evoked excitatory postsynaptic currents (eEPSCs) in TRN neurons were performed by stimulating ChR2-expressing cortical or thalamic axonal terminals. Consistent with the previous studies, the cortico-TRN synaptic response showed paired-pulse facilitation (FIG. 5), confirming the predominant activation of cortico-thalamic descending axonal fibers. In the presence of mGluR2 PAM, AZD8529, significant increase in the paired-pulse ratio (PPR) was observed. These results suggest that the activation of mGluR2 reduced the response of cortico-TRN glutamatergic synapses via a presynaptic mechanism (FIG. 5). No significant changes in PPR in the presence mGluR3 PAM compared to control condition. Thalamo-TRN synaptic responses was measured by activating thalamic axonal terminals. As shown in FIG. 5, it exhibited paired-pulse depression. There were no significant changes in the PPR in the presence of either mGluR2 PAM or mGluR3 PAM. Overall, these results suggest that selective activation of mGluR2 likely reduces the cortical glutamatergic synaptic inputs to TRN neurons. The activation of either mGluR2 or mGluR3 does not show any noticeable effect on thalamo-TRN glutamatergic synapses.

### Example 6: Activation of mGluR_{2/3} Reduces Excitatory Inputs to TRN in Ptchd1 KO Mice

In view of the respective roles of mGluR2 and mGluR3 in differentially regulating TRN circuitry, combinatorial mGluR2/3 treatment for the TRN related dysfunction in Ptchd1 KO mouse model was examined.

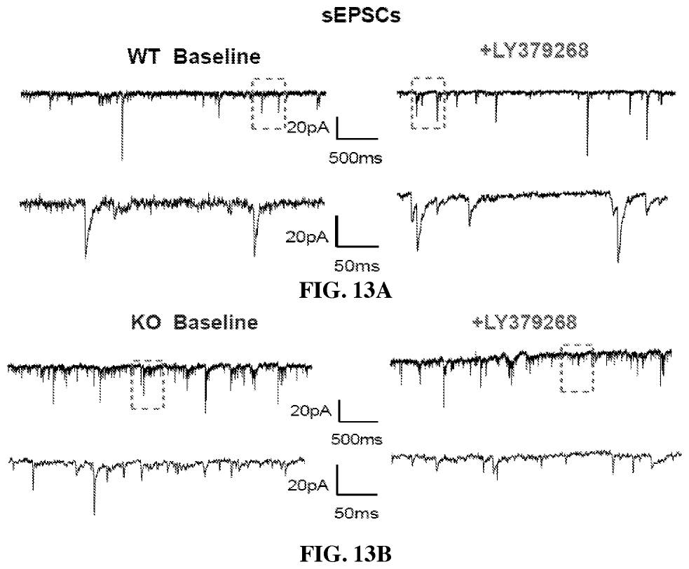

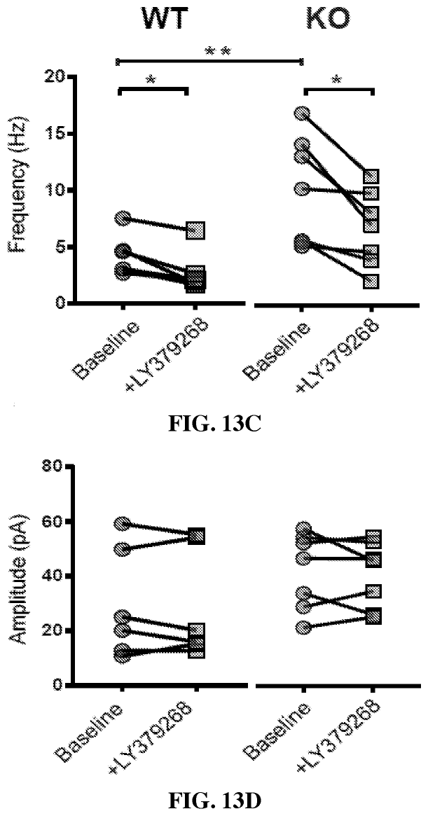

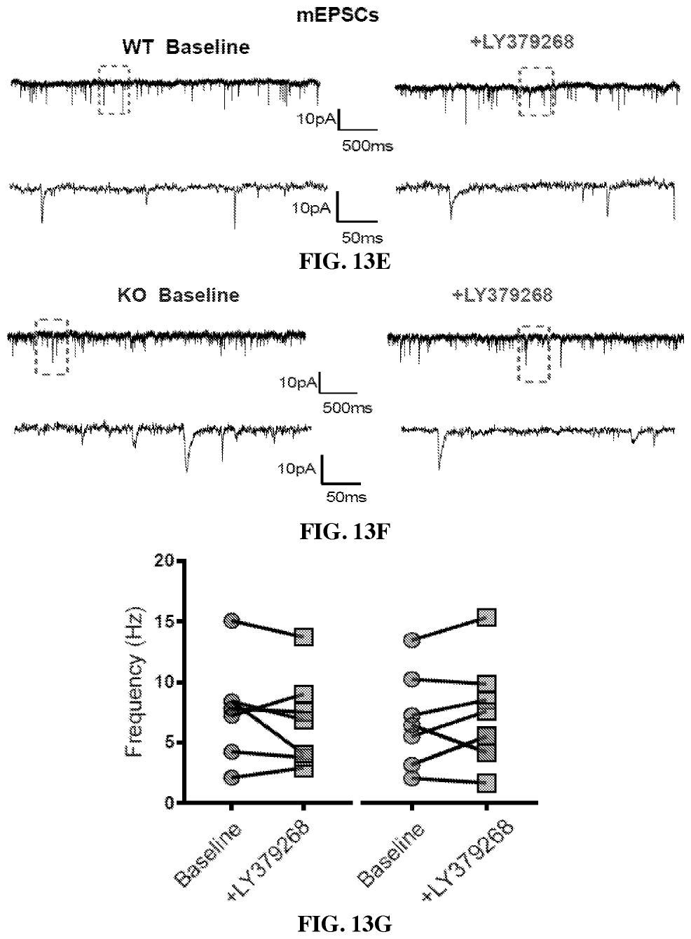

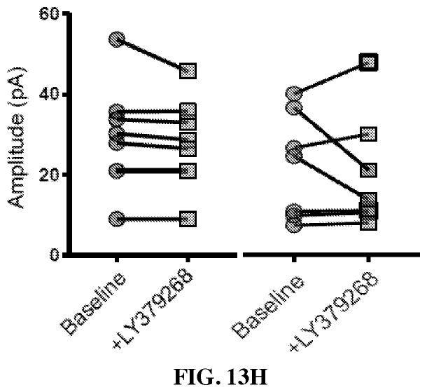

In brief, the synaptic transmission of TRN neurons in this mouse model was characterized. As shown in FIG. 13, whole-cell patch clamp recordings of spontaneous excitatory postsynaptic currents (sEPSCs) from TRN neurons in the absence of TTX showed a significant increase in the frequency, but not peak amplitude in the Ptchd1 KO mice when compared to their age-matched wild type mice. No changes in frequency and peak amplitude of the miniature EPSCs (mEPSCs) in the presence of action potential blocker TTX were observed (FIG. 13E-H). These results suggested an augmented action potential dependent glutamatergic synaptic inputs onto TRN neurons due to the lack of Ptchd1.

Interestingly, the mGluR2/3 agonist LY379268 significantly reduced the frequency, but not amplitude of sEPSCs, in both WT mice and Ptchd1 KO mice, which was consistent with the effect of mGluR2 on reducing presynaptic release probability as shown in FIG. 5. In summary, these results indicate that deficient Ptchd1 causes selective strengthening of the excitatory synapses from cortical inputs, and that type II mGluR activation can normalize hyperexcitability at cortico-TRN synapses.

### Example 7: Activation of mGluR_{2/3} Corrects Sleep and Locomotion Behavioral Deficits in Ptchd1 KO Mouse Model

As shown in the present disclosure, Applicant identified that positive allosteric modulation of mGluR3 facilitates repetitive in Ptchd1 KO mice and corrects fragmented sleep, without affecting cortical or thalamic transmission to the TRN. Applicant also identified that recruitment of mGluR2 via selective agonism of type II mGluRs selectively reduces hyperactive glutamatergic transmission from cortical synapses to the TRN and reduces hyperactivity in our model.

In order to further examine the combined effect of type II mGluR activation on behavioral deficits in the Ptchd1 KO model, the whole-cell patch clamp recordings of TRN neurons showed that the application of the mGluR2/3 agonist LY379268 significantly increased the number of rebound repetitive bursting in Ptchd1 KO TRN neurons at different holding membrane potentials when compared to controls in the absence of LY379268 (FIG. 7A-D). An occlusion experiment was conducted by blocking mGluR3 activity with ML337 before activating mGluR2/3 with LY379268 showed no change in repetitive bursting (FIG. 4) in order to confirm whether burst firing is regulated primarily by mGluR3. Interestingly, ML337 application alone reduced rebound burst firing, suggesting that mGluR3 constitutively regulate rebound burst firing in the TRN (FIG. 4). Furthermore, pretreatment with ML337 blocked LY379268-induced enhancement of rebound bursting (FIG. 4). These results suggest that the mGlu3 activity predominantly contributes to the observed TRN firing activity alterations.

The channel conductance of SK channels and low-threshold T-type Ca2+ channels at different holding membrane potentials ranging from −80 mV to −50 mV in the presence or absence of SK channel blockers was evaluated in order to further determine how the activation of mGluR2/3 in TRN neurons enhanced the rebound bursting. As shown in FIG. 4, both SK and T-type Ca2+ channel conductance were significantly increased in the presence of LY379268 (FIG. 4).

EEG and EMG from the frontal and parietal cortex and from the nuchal muscular complex were simultaneously recorded. As shown in FIG. 7E-7I, the administration of LY379268 (5 mg/kg, i.p.) increased non-REM sleep bout length, in both wild type and Ptchd1 KO mice. These results indicate the capability of LY379268 for improving sleep stability by activation of both mGluR2 and mGluR3 in Ptchd1 KO mice.

The effects of mGluR2/3 activation on the locomotor activity of WT mice and Ptchd1 mutant mice were evaluated. In brief, all mice were treated with mGluR2/3 agonist, LY379268, at three different dosages (1 mg/kg, 5 mg/kg and 10 mg/kg) for open-field testing. Remarkably, as shown in FIG. 7, Ptchd1 KO mice traveled significantly less distances to a comparable level of wild type mice at both 5 mg/kg and 10 mg/kg dosing levels in the open field test. Overall, these results suggest that a combined activation of mGluR2 and mGluR3 rescued both hyperactivity and sleep phenotypes in Ptchd1 KO mouse model.

### Example 8: mGluR2/3 Agonist LY379268 Corrects Sensory Filtering Deficits of Ptchd1 KO Mice

The TRN has been shown to play a key role for sensory filtering by suppressing irrelevant sensory inputs to the thalamic complex. When this circuit is perturbed by deletion of the Ptchd1 gene, animals show increased distractibility, which suggests that they may have diminished sensory filtering. The mechanisms of which remain unclear.

To test whether the activation of mGluR2/3 would rescue the sensory filtering deficits, a behavioral noise filtering test was implemented (Nakajima et al., 2019). Specifically, a cohort of WT and Ptchd1 KO mice were trained to attend to specific auditory stimulus associated with “GO” (tone of 20 KHz) and “NO GO” (tone of 16 and 24 KHz). After a tone was played, mice were required to poke in the response port to get a reward when “GO” tone was played or to withhold poking for 2500 ms when “NO GO” was played (FIG. 11A). Both WT mice and Ptchd1 KO mice showed similar percentage of correct responses while they were performing without noise interference (FIGS. 11C and 11D). Significant reduction in performance was observed in Ptchd1 KO mice in the presence of the auditory stimulus embedded with noise, as expected in view of the dysfunctional TRN. The administration of mGluR2/3 LY379268 (5 mg/kg i.p.) fully rescued noise filtering deficits in the Ptchd1 KO to control level.

### Example 9: mGluR3 PAM Treatment Corrects Sleep Disruption in Ptchd1 KO Mouse Model

The TRN mediated thalamo-cortical circuit is considered to be one of the main brain circuits involving the generation of cortical rhythms (Crunelli V & Hughes S W, 2010). Studies have suggested a causal relationship between TRN burst firing activity and sleep rhythms. Applicant's study showed that sleep fragmentation was exhibited in the Ptchd1 KO mouse model (Wells et al., 2016). The effect of mGluR₃ activation on TRN repetitive bursting would predict a modulation of the sleep architecture and thalamo-cortical rhythms in Ptchd1 KO mouse model.

Surface electroencephalography (EEG) and electromyography (EMG) from the frontal and parietal cortex and from the nuchal muscular complex were simultaneously recorded. As shown in FIG. 9, the administration of Mavalon 63 (10 mg/kg, intraperitoneal injection, i.p.) specifically increased the Non-REM (NREM) sleep bout length in and Ptchd1 KO mice, but not wild type mice (FIG. 9). These results support a potential improvement in sleep stability by selective activation of mGluR3, which is consistent with the ability of Mavalon 63 to enhance TRN rebound burst firing.

### Example 10: mGluR2 PAM Corrects Hyperactivity of Ptchd1 KO Mice

Distractibility is often accompanied by hyperactivity in several neurodevelopmental disorders. ADHD symptoms are frequently observed in patients with PTCHD1 mutations. It was previously shown that the Ptchd1 KO mice exhibited a hyperactivity phenotype in the open-field behavioral test (Wells et al., 2016).

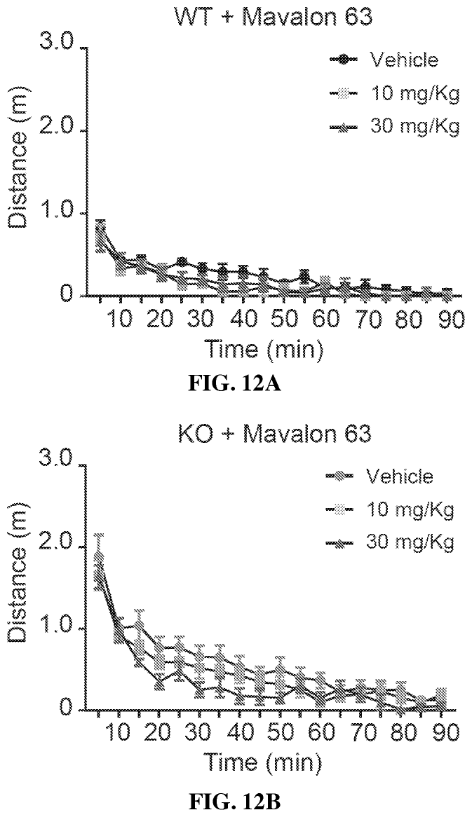

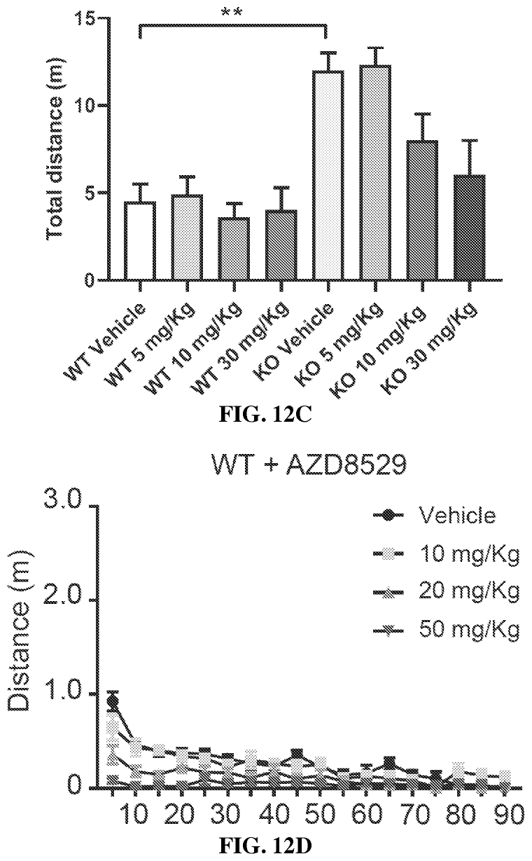

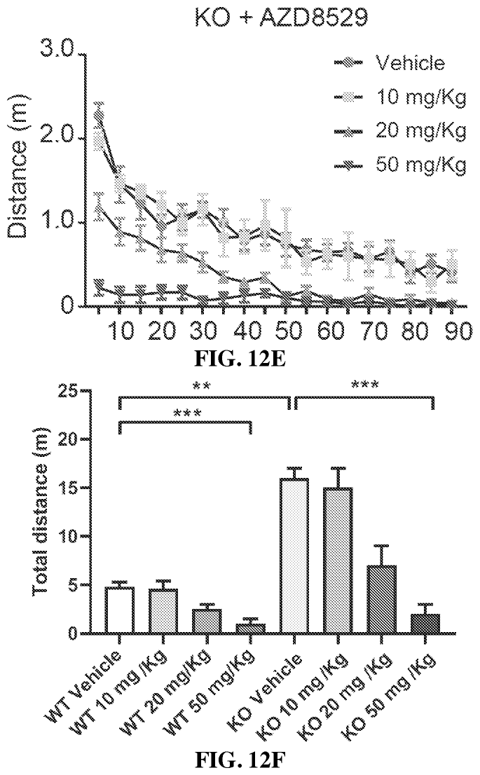

Here, whether mGluR3 activation would rescue the hyperactivity phenotype of Ptchd1 KO mice was evaluated. Locomotor activity estimated as total distance travelled (in) was assessed over a 90 min period in an automated Omnitech Digiscan apparatus. For drug treatment experiments, mice were placed in the open-field arena for 30 minutes prior to Mavalon 63 (5 mg/kg and 10 mg/kg in saline; i.p.), or vehicle injections before being returned to the arena. Mavalon 63 had no significant effect on locomotor activity in WT mice and their age-matched Ptchd1 KO mice (FIG. 12A-12C). However, a trend of reducing the hyperactivity of Ptchd1 KO mice compared to control mice (p=0.06, Bonferroni's multiple comparisons test) was observed when reviewing the locomotor activity of Ptchd1 KO mice treated with a higher dose at 30 mg/kg (i.p.). No effect on the spontaneous locomotion activity for wild type mice treated with the high dose was observed (FIGS. 12A and 12C).

It is observed that Mavalon 63 was able to normalize the hyperactivity phenotype of Ptchd1 KO mice, but not WT mice, in a dose-dependent manner. Here, Applicant hypothesized that other mGluR₃-related molecular substrates would similarly determine circuit endophenotypes of hyperactivity. GroupII mGluR activation has consistently been shown to reduce locomotor activity in context of both spontaneous and dopaminergic challenged conditions (Moghaddam & Adams, 1998; Spooren, 2000, Pehrson & Moghaddam et al, 2010, Boerner et al., 2017 Wooden et al., 2018). Since Group II mGluRs comprise both mGluR2 and mGluR3, Applicant reasoned that the hyperlocomotion regulation could rely on mGluR2 activity in Ptchd1 KO mouse model. To that end, the locomotor activity of WT and Ptchd1 KO mice treated with mGluR2 PAM and AZD8529, at three different dosing levels for open-field testing was evaluated. Remarkably, Applicant found that AZD8529 reduced locomotor activity in both WT and hyperactive Ptchd1KO mice in a dose-dependent manner in the open field test (FIG. 12D-12F). Without wishing to be bound by any theory, these results suggest that mGluR2 and mGluR3 may differentially regulate the hyperactivity phenotype in Ptchd1 KO mouse model by targeting different TRN mediated circuit components.

### REFERENCES

Agmon, A., & Connors, B. W. (1991). Thalamocortical responses of mouse somatosensory (barrel) cortexin vitro. Neuroscience, 41(2-3), 365-379.
Augier, E., Dulman, R. S., Rauffenbart, C., Augier, G., Cross, A., & Heilig, M. (2016) The mGluR2 Positive Allosteric Modulator, AZD8529, and Cue-Induced Relapse to Alcohol Seeking in Rats. Neuropsychopharmacology, 41, 2932-2940
Boerner, T., Bygrave, A. M., Sanderson, D. J. (2017) The group II metabotropic glutamate receptor agonist LY354740 and the D2 receptor antagonist haloperidol reduce locomotor hyperactivity but fail to rescue spatial working memory in GluA1 knockout mice. Eur J Neurosci. 45(7): 912-921.
Bourassa, J., Pinault, D., & Deschênes, M. (1995). Corticothalamic projections from the cortical barrel field to the somatosensory thalamus in rats: a single-fibre study using biocytin as an anterograde tracer. European journal of neuroscience, 7(1), 19-30.
Crandall, S. R., Cruikshank, S. J., & Connors, B. W. (2015). A corticothalamic switch: controlling the thalamus with dynamic synapses. Neuron, 86(3), 768-782.
Cruikshank, S. J., Urabe, H., Nurmikko, A. V., & Connors, B. W. (2010). Pathway-specific feedforward circuits between thalamus and neocortex revealed by selective optical stimulation of axons. Neuron, 65(2), 230-245.
Crunelli V. & Hughes S. W. (2010) The slow (<1 Hz) rhythm of non-REM sleep: a dialogue between three cardinal oscillators. Nat Neurosci. 13(1):9-17. doi: 10.1038/nn.2445.
Ferraguti, F. & Shigemoto R., (2006) Metabotropic gluatmate receptors. Cell and Tissue Research, 326 (2), 483-504.
Fogerson, P. M., & Huguenard, J. R. (2016). Tapping the brakes: cellular and synaptic mechanisms that regulate thalamic oscillations. Neuron, 92(4), 687-704.
Gentet, L. J., & Ulrich, D. (2003). Strong, reliable and precise synaptic connections between thalamic relay cells and neurones of the nucleus reticularis in juvenile rats. The Journal of physiology, 546(3), 801-811.
Gu, G., Lorrain, D. S., Wei, H., Cole, R. L., Zhang, X., Daggett, L. P., Schaffhauser, H. J., Bristow, L. J., Lechner, S. M. (2008) Distribution of metabotropic glutamate 2 and 3 receptors in the rat forebrain: implication in emotional responses and central disinhibition. Brain Res. 1197, 47-62
Habib, N., Li, Y., Heidenreich, M., Swiech, L., Avraham-Davidi, I., Trombetta, J. J. & Regev, A. (2016). Div-Seq: Single-nucleus RNA-Seq reveals dynamics of rare adult newborn neurons. Science, 353(6302), 925-928.
Harrison P J I, Lyon L, Sartorius L J, Burnet P W, Lane T A. (2008) The group II metabotropic glutamate receptor 3 (mGluR3, mGlu3, GRM3): expression, function and involvement in schizophrenia. J Psychopharmacol 22(3):308-22.
Jurgens, C. W., Bell, K. A., McQuiston, A. R., & Guido, W. (2012). Optogenetic stimulation of the corticothalamic pathway affects relay cells and GABAergic neurons differently in the mouse visual thalamus. Plos one, 7(9), e45717.
Kaiser, T., Ting, J. T., Monteiro, P., & Feng, G. (2016). Transgenic labeling of parvalbumin-expressing neurons with tdTomato. Neuroscience, 321, 236-245.
Landisman, C. E., & Connors, B. W. (2007). VPM and PoM nuclei of the rat somatosensory thalamus: intrinsic neuronal properties and corticothalamic feedback. Cerebral cortex, 17(12), 2853-2865.
Lam, Y. W., & Sherman, S. M. (2011). Functional organization of the thalamic input to the thalamic reticular nucleus. Journal of Neuroscience, 31(18), 6791-6799.
Pinault, D. (2004). The thalamic reticular nucleus: structure, function and concept. Brain research reviews, 46(1), 1-31.
Wells M F, Wimmer R D, Schmitt L I, Feng G, Halassa M M (2016) Thalamic reticular impairment underlies attention deficit in Ptchd1(Y/−) mice. Nature, 7597, 58.
Chaudhry A, Noor A, Degagne B, Baker K. et al. (2015) Phenotypic spectrum associated with PTCHD1 deletions and truncating mutations includes intellectual disability and autism spectrum disorder. Clin Genet. 88:224-233.
Noor A, Whibley A, Marshall C R, Gianakopoulos P J, Piton A, Carson A R et al. Disruption at the PTCHD1 Locus on Xp22.11 in Autism spectrum disorder and intellectual disability. Sci Transl Med 2010; 2: 49ra68.
Torrico B, Fernandez-Castillo N, Hervis A, Mili M, Salgado M, Rueda I et al. Contribution of common and rare variants of the PTCHD1 gene to autism spectrum disorders and intellectual disability. Eur J Hum Genet 2015; 23: 1694-1701.
Moghaddam B I, Adams B W. (1998) Reversal of phencyclidine effects by a group II metabotropic glutamate receptor agonist in rats. Science 281(5381): 1349-1352
Neki A, Ohishi H, Kaneko T, Shigemoto R, Nakanishi S, Mizuno N (1996) Pre- and postsynaptic localization of a metabotropic glutamate receptor, mGluR2, in the rat brain: an immunohistochemical study with a monoclonal antibody. Neurosci Lett. 202(3):197-200.
Alan Pehrson, Bita Moghaddam (2010) Impact of metabotropic glutamate 2/3 receptor stimulation on activated dopamine release and locomotion. Psychopharmacology 211(4):443-455
Petralia R S, Wang Y X, Niedzielski A S, Wenthold R J (1996) The metabotropic glutamate receptors, mGluR2 and mGluR3, show unique postsynaptic, presynaptic and glial localizations. Neuroscience. 71(4):949-76.
Spooren W. P. (2000) Lack of effect of LY314582 (a group 2 metabotropic glutamate receptor agonist) on phencyclidine-induced locomotor activity in metabotropic glutamate receptor 2 knockout mice. Eur. J. Pharmacol. 397(1):R1-R2
Spooren W, Ballard T, Gasparini F, Amalric M, Mutel V, Schreiber R. (2003) Insight into the function of Group I and Group II metabotropic glutamate (mGlu) receptors: behavioural characterization and implications for the treatment of CNS disorders. Behav Pharmacol. 14:257-277.
Wood C M, Wafford K A, McCarthy A P, Hewes N, Shanks E, Lodge D, Robinson E S J. (2018) Investigating the role of mGluR₂ versus mGluR₃ in antipsychotic-like effects, sleep-wake architecture and network oscillatory activity using novel Han Wistar rats lacking mGluR₂ expression. Neuropharmacology. 140:246-259. doi: 10.1016/j.neuropharm.2018.07.013.
Wright R A, Arnold M B, Wheeler W J, Ornstein P L, Schoepp D D (2001) LY341495 binding to group II metabotropic glutamate receptors in rat brain. J Pharmacol Exp Ther, 298:453-460.

### EQUIVALENTS

Those skilled in the art will recognize, or be able to ascertain using no more than routine experimentation, many equivalents to the specific embodiments of the invention described herein. Such equivalents are intended to be encompassed by the following claims.

1. A method for treating a neurodevelopmental disorder, the method comprising administering to a subject in need thereof an effective amount of a group II metabotropic glutamate receptor modulator.

2. The method of claim 1, wherein the group II metabotropic glutamate receptor modulator is a mGluR_{2/3} agonist.

3. The method of claim 2, wherein the mGluR_{2/3} agonist is selected from the group consisting of LY354740, MGS0028, LY379268, LY2934747, LY2969822, LY404040, LY404039, and LY2140023.

4. The method of claim 1, wherein the group II metabotropic glutamate receptor modulator is a mGluR₃-specific modulator.

5. The method of claim 4, wherein the mGluR₃-specific modulator is a mGluR₃ agonist or a mGluR₃ positive allosteric modulator (PAM).

6. The method of claim 5, wherein the mGluR₃ agonist is LY2794193.

7. The method of claim 5, wherein the mGluR₃ PAM is selected from the group consisting of DT011088, Mavalon-63 PAM, and Mavalon-207 PAM.

8. The method of any one of claims 1-7, wherein the neurodevelopmental disorder is selected from the group consisting of: attention-deficit hyperactivity disorder (ADHD), a learning disorder, a motor disorder, a tic disorder, a speech disorder, a genetic disorder, a neurotoxicants-related disorders, intellectual disability (ID), and an autism spectrum disorder (ASD).

9. The method of any one of claims 1-7, wherein the subject is a human subject having, suspected of having, or at risk of developing a neurodevelopmental disorder selected from the group consisting of: attention-deficit hyperactivity disorder (ADHD), a learning disorder, a motor disorder, a tic disorder, a speech disorder, a genetic disorder, a neurotoxicants-related disorders, intellectual disability (ID), or an autism spectrum disorder (ASD).

10. The method of any one of claims 1-9, wherein the subject is a human subject having a loss-of-function mutation in the Patched Domain Containing 1 (PTCHD1) gene.

11. The method of any one of claims 1-10 further comprising administering to the subject an additional therapeutic agent.

12. The method of claim 11, wherein the additional therapeutic agent is selected from the group consisting of metformin, memantine, flumazenil, meclofenoxate, risperidone, carbamazepine, sodium valproate, lamotrigine, lithium carbonate, methylphenidate, procyclidine, ferrous fumarate+vitamins+lactulose+cod liver oil+various skin ointments, clobazam+lorazepam, rectal diazepam+buccal midazolam, omega-3 fatty acids+inositol, n-acetylcysteine, intranasal oxytocin, memantine hydrochloride, lovastatin, BPN14770, dronabinol, THC, 18F-AV-1451, ketamine, midazolam, d-cycloserine, rivaroxaban, acetylsalicylic acid, metformin amisulpride, bromocriptine, acetazolamide, antipsychotics including risperidone, aripiprazole, ziprasidone, SSRIs including fluoxetine, citalopram, escitalopram, stimulants including methylphenidate, alpha-2-adrenergic agonists including clonidine, guanfacine, propranolol, beta-blockers, primidone, clonazepam, diazepam, lorazepam, alprazolam, dopamine antagonists, aripiprazole, antipsychotics including clonidine, risperidone, olanzapine, ziprasidone, haloperidol, fluphenazine, pimozide, tetrabenazine, guanfacine, skeletal muscle relaxants including baclofen, benzodiazepines including clonazepam, neuromuscular blockers including onabotulinumtoxinA, amantadine, cyclosporine A, donepezil, glyburide, lithium, methylphenidate, minocycline, progesterone, rivastigmine, simvastatin, lithium, fenobam, γ-aminobutyric acid agonists, analgesics including acetametophin and codeine, morphine, ibuprofen, naproxen, antidysrthymics including digoxin, furosemide, hydrochlorothiazide, metolazone, memantine, antipsychotics including aripiprazole, asenapine, brexpiprazole, buspirone, cariprazine, chlorpromazine hydrochloride, clozapine, haloperidol, iloperidone, loxapine, lumateperone, lurasidone hydrochloride, molindone hydrochloride, olanzapine, paliperidone, perphenazine, prochlorperazine, quetiapine, risperidone, thiothixene, trifluoperazine, ziprasidone, androgens including testosterone,methyltestosterone, fluoxymesteron, gonadotrophins including chorionic gonadotropin and follitropin, teriparatide, stimulants including methamphetamine, methylphenidate, dexmethylphenidate, amphetamine, dextroamphetamine, Lisdexamfetamine, isdexamfetamine dimesylate, bupropion, venlafaxine, imipramine, risperidone, lithium and methylphenidate, carbamazepine, quetiapine, anticonvulsants such as citalopram, escitalopram, fluoxetine, paroxetine, sertraline, central alpha-2 adrenergic agonists including clonidine, guanfacine, and norepinephrine reuptake inhibitors including atomoxetine.

13. A method for treating attention-deficit hyperactivity disorder (ADHD), comprising administering an effective amount of a group II metabotropic glutamate receptor modulator to a subject who has been diagnosed with ADHD.

14. A method for treating a psychiatric disorder the method comprising administering to a subject in need thereof an effective amount of a group II metabotropic glutamate receptor modulator, wherein the psychiatric disorder is not schizophrenia.

15. A method for treating a sleep disorder the method comprising administering to a subject in need thereof an effective amount of a group II metabotropic glutamate receptor modulator.

16. A method of improving sleep quality and/or increasing sleep duration in a subject comprising administering to the subject an effective amount of a group II metabotropic glutamate receptor modulator.

17. The method of any one of claims 13-16, wherein the group II metabotropic glutamate receptor modulator is a mGluR_{2/3} agonist.

18. The method of claim 17, wherein the mGluR_{2/3} agonist is selected from the group consisting of LY354740, MGS0028, LY379268, LY2934747, LY2969822, LY404040, LY404039, and LY2140023.

19. The method of any one of claims 13-16, wherein the group II metabotropic glutamate receptor modulator is a mGluR₃-specific modulator.

20. The method of claim 19, wherein the mGluR₃-specific modulator is a mGluR₃ agonist or a mGluR₃ positive allosteric modulator (PAM).

21. The method of claim 20, wherein the mGluR₃ agonist is a LY2794193.

22. The method of claim 20, wherein the mGluR₃ PAM is selected from the group consisting of DT011088, Mavalon-63 PAM, and Mavalon-207 PAM.

23. The method of any one of claims 13-22, wherein the subject is a human subject.

24. A method for treating a neurodevelopmental disorder, the method comprising: administering an effective amount of a group II metabotropic glutamate receptor modulator to a subject who has been identified as having a loss-of-function mutation in the Patched Domain Containing 1 (PTCHD1) gene.

25. A method for treating a neurodevelopmental disorder, the method comprising:
determining whether a subject has a loss-of-function mutation in the Patched Domain Containing 1 (PTCHD1) gene, and
administering an effective amount of a group II metabotropic glutamate receptor modulator to the subject if the subject has a loss-of-function mutation in the PTCHD1 gene.

26. The method of claim 24, wherein a subject is identified as having a loss-of-function mutation in the PTCHD1 gene based on a polymerase chain reaction (PCR) assay and/or a nucleic acid microarray assay.

27. The method of claim 25, wherein determining whether a subject has a loss-of-function mutation in the PTCHD1 gene comprises conducting a polymerase chain reaction (PCR) assay and/or a nucleic acid microarray assay.

28. The method of claim 24, wherein a subject is identified as having a loss-of-function mutation in the Patched Domain Containing 1 (PTCHD1) gene based on an immunohistochemical assay, an immunoblotting assay, and/or a flow cytometry assay.

29. The method of claim 25, wherein determining whether a subject has a loss-of-function mutation in the PTCHD1 gene comprises conducting an immunohistochemical assay, an immunoblotting assay, and/or a flow cytometry assay.

30. The method of any one of claims 24-29, wherein the loss-of-function mutation in the PTCHD1 gene is selected from the group consisting of an insertion, a deletion, and a substitution.

31. The method of any one of claims 24-30, wherein the group II metabotropic glutamate receptor modulator is a mGlu_{2/3} agonist.

32. The method of claim 31, wherein the mGlu_{2/3} agonist is selected from the group consisting of LY354740, MGS0028, LY379268, LY2934747, LY2969822, LY404040, LY404039, and LY2140023.

33. The method of any one of claims 24-30, wherein the group II metabotropic glutamate receptor modulator is an mGlu₃-specific modulator.

34. The method of claim 33, wherein the mGlu₃-specific modulator is a mGlu₃ agonist or a mGlu₃ positive allosteric modulator (PAM).

35. The method of claim 34, wherein the mGlu₃ agonist is LY2794193.

36. The method of claim 34, wherein the mGlu₃ PAM is selected from the group consisting of DT011088, Mavalon-63 PAM, and Mavalon-207 PAM.

37. The method of any one of claims 24-36, wherein the subject is a human subject having, suspected of having, or at risk of developing a neurodevelopmental disorder.

38. The method of claim 37, wherein the neurodevelopmental disorder is selected from the group consisting of: attention-deficit hyperactivity disorder (ADHD), a learning disorder, a motor disorder, a tic disorder, a speech disorder, a genetic disorder, a neurotoxicants-related disorders, intellectual disability (ID), or an autism spectrum disorder (ASD).

39. The method of any one of claims 24-38, further comprising administering to the subject an additional therapeutic agent.

40. The method of claim 39, wherein the additional therapeutic agent is selected from the group consisting of metformin, memantine, flumazenil, meclofenoxate, risperidone, carbamazepine, sodium valproate, lamotrigine, lithium carbonate, methylphenidate, procyclidine, ferrous fumarate+vitamins+lactulose+cod liver oil+various skin ointments, clobazam+lorazepam, rectal diazepam+buccal midazolam, omega-3 fatty acids+inositol, n-acetylcysteine, intranasal oxytocin, memantine hydrochloride, lovastatin, BPN14770, dronabinol, THC, 18F-AV-1451, ketamine, midazolam, d-cycloserine, rivaroxaban, acetylsalicylic acid, metformin amisulpride, bromocriptine, acetazolamide, antipsychotics including risperidone, aripiprazole, ziprasidone, SSRIs including fluoxetine, citalopram, escitalopram, stimulants including methylphenidate, alpha-2-adrenergic agonists including clonidine, guanfacine, propranolol, beta-blockers, primidone, clonazepam, diazepam, lorazepam, alprazolam, dopamine antagonists, aripiprazole, antipsychotics including clonidine, risperidone, olanzapine, ziprasidone, haloperidol, fluphenazine, pimozide, tetrabenazine, guanfacine, skeletal muscle relaxants including baclofen, benzodiazepines including clonazepam, neuromuscular blockers including onabotulinumtoxinA, amantadine, cyclosporine A, donepezil, glyburide, lithium, methylphenidate, minocycline, progesterone, rivastigmine, simvastatin, lithium, fenobam, γ-aminobutyric acid agonists, analgesics including acetametophin and codeine, morphine, ibuprofen, naproxen, antidysrthymics including digoxin, furosemide, hydrochlorothiazide, metolazone, memantine, antipsychotics including aripiprazole, asenapine, brexpiprazole, buspirone, cariprazine, chlorpromazine hydrochloride, clozapine, haloperidol, iloperidone, loxapine, lumateperone, lurasidone hydrochloride, molindone hydrochloride, olanzapine, paliperidone, perphenazine, prochlorperazine, quetiapine, risperidone, thiothixene, trifluoperazine, ziprasidone, androgens including testosterone,methyltestosterone, fluoxymesteron, gonadotrophins including chorionic gonadotropin and follitropin, teriparatide, stimulants including methamphetamine, methylphenidate, dexmethylphenidate, amphetamine, dextroamphetamine, Lisdexamfetamine, isdexamfetamine dimesylate, bupropion, venlafaxine, imipramine, risperidone, lithium and methylphenidate, carbamazepine, quetiapine, anticonvulsants such as citalopram, escitalopram, fluoxetine, paroxetine, sertraline, central alpha-2 adrenergic agonists including clonidine, guanfacine, and norepinephrine reuptake inhibitors including atomoxetine.
